(19) United States
(12) Patent Application Publication
(10) Pub. No.: US 2024/0312631 A1
(43) Pub. Date: Sep. 19, 2024

(54) ASSESSING PATIENT HEALTH RISKS & PREDICTING HEALTH EVENTS USING BIOSIGNALS FROM SMARTPHONES & OTHER DEVICES

(71) Applicant: Measure Labs, Inc., Seattle, WA (US)

(72) Inventors: Matthew Steven Whitehill, Seattle, WA (US); Jamien McCullum, Seattle, WA (US); Leon Alexander Gatys, Seattle, WA (US); Ryan Edward Young, Seattle, WA (US)

(21) Appl. No.: 18/586,205

(22) Filed: Feb. 23, 2024

Related U.S. Application Data
(60) Provisional application No. 63/486,627, filed on Feb. 23, 2023.

(51) Int. Cl. G16H 50/20; A61B 5/022; G16H 10/60; G16H 50/30

(52) U.S. Cl. CPC G16H 50/20; A61B 5/022; G16H 10/60; G16H 50/30

(57) ABSTRACT

Techniques and systems include assessing health risk and predicting health events of a patient using measured biosignals. The biosignals may be transformed into a photoplethysmography (PPG) signal or pseudo PPG signal, for example, to measure blood volume changes in the patient's blood flow to derive data indicating a disease state or health-related characteristic, such as blood oxygen level, blood glucose level, heart rate variability, hemoglobin, respiration rate, or arrhythmia. Further, the biosignals can include vocal signals, health labels, and self-reported data related to a patient's health. Assessment and prediction techniques include training an artificial intelligence (AI) model to create embedding vectors based on the measured biosignal(s) that are then ingested by a prediction model to assess and/or predict a patient's health risk and/or health events.

### CROSS-REFERENCE TO RELATED APPLICATIONS

This application claims priority and benefit from the U.S. Provisional Patent Application No. 63/486,627, filed Feb. 23, 2023, and titled, “ASSESSING PATIENT HEALTH RISK USING PPG SIGNALS FROM SMARTPHONES,” which is incorporated herein by reference in its entirety for all purposes.

### BACKGROUND

There are several quantities that are regularly measured and evaluated to provide information about a person's health. These quantities and both their real-time values and trends over various timescales can sometimes be used as indications of disease or a serious health condition. Examples of such quantities that may be used to monitor and, in some cases, diagnose or predict a health condition, risk, or future event. Such quantities can include blood pressure, pulse or heart rate, and blood oxygen saturation (SpO2), among other biomarkers, the presence of certain health conditions like hypertension or diabetes, or medical events like heart attack or stroke. Conventionally, such health-related quantities are measured at a health clinic, the office of a provider of medical services, or with special equipment that is used in the home, which can be time-consuming, costly, confusing to patients, and burdensome.

Notably, these conventional methods of measuring health quantities are not always available, may require advance planning to use, and are not convenient for situations where a person is engaging in activities away from home or must operate complicated medical devices at home. Further, the conventional methods provide a “snapshot” in time of the values of the health-related quantities but may not provide sufficient data or data collected at a sufficient time or set of times to detect or diagnose a health condition. For example, a physician or paramedic may want to know a person's blood pressure over a time interval around when they suffered some type of pain. This may be difficult to do without the person being in a location where someone can accurately make those measurements. As another example, a person or the person's medical provider may want to monitor how their blood pressure responds to certain activities or behaviors for various reasons, such as overall health, predicting health risk and events, or monitoring for specific disease states or conditions, and such monitoring may be difficult to do outside of their home.

Although there are some portable devices that can be used to monitor blood pressure and other biomarkers, these typically are not as accurate as desired. For example, although portable blood pressure monitors are available, these typically require that a person be in a situation where a “cuff” may be used to obtain the blood pressure reading, and this is not always possible or convenient and may result in poor data acquisition due to the blood pressure cuff operation by the patient.

What is desired are systems, apparatuses, and methods for enabling a person and/or a person's medical provider(s) to assess a patient's physiological state and predict health conditions and health events.

### BRIEF DESCRIPTION OF THE DRAWINGS

The disclosure will be understood more fully from the detailed description given below and from the accompanying figures of embodiments of the disclosure. The figures are used to provide knowledge and understanding of embodiments of the disclosure and do not limit the scope of the disclosure to these specific embodiments.

FIG. 1 illustrates a user engaging a camera of a smartphone for measuring a biomarker or other health-related quantity, according to some embodiments.

FIG. 2 illustrates example photoplethysmography (PPG) signal waveforms and a comparison between a PPG signal obtained from a pulse oximeter and a signal derived from the red channel of an RGB video signal, according to some embodiments.

FIG. 3 is a block diagram illustrating a process flow and functional elements that may be used in implementing various embodiments.

FIG. 4 illustrates a process that uses a baseline model incorporating an averaged red video channel to create a time-series signal that resembles a PPG signal, according to some embodiments.

FIG. 5 illustrates a process that uses a baseline model incorporating a time-series of video frames that are passed directly to a deep learning model to predict a biomarker, according to some embodiments.

FIG. 6 is a flow diagram of a process for addressing signal quality of a PPG signal, according to some embodiments.

FIG. 7 is a flow diagram of a process for determining signal quality of a PPG signal and determining a disease state or biomarker based on the PPG signal, according to some embodiments.

FIG. 8 illustrates elements or components of a computer device or system configured to implement a method, process, function, or operation of some embodiments described herein.

FIG. 9 illustrates a Software-as-a-Service (SaaS) system in which some embodiments described herein may be implemented.

FIG. 10 illustrates elements or components of an example operating environment in which some embodiments described herein may be implemented.

FIG. 11 illustrates some details of elements or components of a multi-tenant distributed computing service platform, of which some embodiments described herein may be implemented.

FIGS. 12A & 12B illustrate actual v. estimated risk for various health conditions based on embodiments disclosed herein.

FIG. 13A is a block diagram illustrating a process of training a patient embedding vector model on reference patient data.

FIG. 13B is a block diagram illustrating a process flow and functional elements that may be used in implementing various embodiments disclosed herein relating to assessing patient health risk and predicting health events using biosignals.

FIG. 14 illustrates a deep learning embedding model trained by receiving a biosignal, such as a PPG signal, which is condensed to an embedding vector that provides a numerical representation of a patient's physiological health.

FIG. 15 illustrates a health risk/event prediction model that uses the embedding vector produced by the deep learning embedding model shown in FIG. 14 to assess a patient's health risk or predict health events based on a nearest neighbor.

FIG. 16 is a flow diagram of a process for using creating an embedding vector for a patient biosignal to assess a patient health risk or predict a patient health event based on a nearest neighbor model.

FIG. 17 is a flow diagram of a process of assessing a patient's health risks and predicting events using a temporal pattern or temporal embedding.

### DETAILED DESCRIPTION

This disclosure describes a number of techniques and systems for determining or predicting various health conditions, health events, disease states, or other health factors related to a patient to help patients and medical providers intervene with preventive healthcare when needed. This predictive healthcare approach helps reduce cost and improve patient outcomes by using data to identify risk and intervene before a patient's health issue escalates into serious conditions or the patient experiences expensive medical events, all of which can have high morbidity and mortality rates that can severely impact patients' lives, their families and communities, and strain healthcare providers and systems. Conventionally, predictive healthcare rarely existed or was unreliable because it relies on robust, current, and available health data about a patient. Most patient data in the conventional system is sparse, outdated, or simply unavailable, which makes predictive healthcare difficult or nearly impossible to provide patients. Without such data, predictive healthcare risk estimations and patient health assessments are unreliable at best and remain only available to those patients in frequent contact with a healthcare system.

Predictive healthcare involves using data to estimate a patient's individual risk of developing a certain condition or having a condition progress and providing responsive interventions to prevent or manage the condition. The disclosed techniques and systems involve use of one or more biosignals, such as an optical signal like a PPG signal, a video signal transformed into a pseudo PPG signal, or a vocal signal that are processed using a deep learning model. The deep learning model extracts an embedding vector that includes a numerical representation of a patient's physiological state, which becomes a “physiological signature” or “physiological fingerprint” of the patient at the time of the measurement. The embedding vector is a numerical representation that summarizes the physiological information in the subject biosignal and represents the physiological health of a person at a given moment in time. The physiological signature of the biosignal(s) or embedding vector(s) of the target patient are compared to the physiological signatures or embedding vectors, respectively, of the biosignal(s) of one or more reference patients.

For example, a single embedding vector can be a patient's age, weight, and height determined from a patient's PPG signal and/or a vocal signal, which are measured from different sources. The different sources then transmit the respective signals to be used to create respective embedding vector(s). The patient's embedding vector based on their PPG signal is compared to the embedding vector(s) based on the PPG signal of the reference patients. Similarly, the patient's embedding vector based on their vocal signal is compared to the embedding vector based on the reference patient(s) vocal signal. The deep learning model condenses each measured biosignal(s) into a concise numerical form, which is an embedding vector that contains useful physiological information to help assess a patient's health conditions and predict health events.

Multiple measurements can create a physiological signature of the patient as their health condition changes over time. This physiological signature can be represented with a target patient embedding vector. Multiple measurements can be taken from any one or more sources at any desired interval, and the interval at which the measurements are taken can target a specific health condition to monitor over time in some examples. Specifically, a patient being monitored for diabetes may have their measurements taken multiple times a day or multiple times a week while someone being monitored for high cholesterol or heart disease may have measurements taken weekly or twice a month. A patient can be monitored for a single condition or multiple conditions, as needed.

Any one or a combination of the measured biosignals can be used to derive data indicating a disease state, biomarker, or other physiological indicator. The disease state, biomarker, or other physiological indicator are used to assess the patient's health risk(s) and predict health events, such as risk of hypertension, diabetes, high cholesterol, heart disease, and high blood pressure, among other health conditions. In the past, an optical signal used for this purpose could suffer from quality and inconsistency issues, even in a controlled environment on the same patient. Such an optical signal was not combined with other biosignals, such as a vocal signal or other physiological indicator to assess patient health risks and predict health events.

One technique for assessing patient's health risks and predicting health events uses photoplethysmography (PPG) measurement(s) of a patient. PPG is an optical technique used to detect volumetric changes in blood in peripheral circulation, using low-intensity infrared (IR) light. When light travels through biological tissues it is absorbed by bones, skin pigments, and both venous and arterial blood. Since light is more strongly absorbed by blood than surrounding tissues, changes in blood flow can be detected by PPG sensors as indicated by changes in the transmitted intensity of light. A voltage signal from PPG sensors may be proportional to the quantity of blood flowing through blood vessels. Even small changes in blood volume can be detected using this method. A PPG signal has several components including volumetric changes in arterial blood that is associated with cardiac activity, variations in venous blood volume that modulates the PPG signal, and a DC component showing the tissues' optical properties and subtle energy changes in the body. In some examples, this change is observed by the camera in 5 millisecond windows.

A PPG signal can also increase, decrease, or otherwise show changes in signal features or characteristics such as amplitude, smoothness or shape of a signal curve, frequency, and/or phase in response to changes or indicators in blood pressure, blood glucose levels (A1c), vascular age, atherosclerosis, respiratory system health, and nervous system health, and other health conditions and characteristics. These changes or indicators help determine a physiological health state for a patient, which can help patients, medical providers, and others assess a patient's health and assess health risks and predict health events with either a single measurement or multiple measurements over time. A single measurement is useful information, particularly during screening for different health conditions like hypertension, diabetes, or heart disease, to determine if full diagnostic tests or other treatment or preventive actions should take place. However, when multiple measurements of the PPG signal are analyzed, a patient's physiological state can be analyzed over that time period to help the patient and their medical provider assess their health risks and predict health events, which give them the opportunity to intervene and treat a patient early in the risk or disease development or progression. This data could be integrated into an electronic health record (EHR) or pushed to medical providers and/or insurance companies to better identify rising risk for early intervention by a provider or care team.

Previous efforts to derive blood pressure or other health metrics from smartphone-sourced PPG have shown promising results in lab settings but have not reached good enough accuracy in real-world settings for widespread adoption. A substantial technical barrier to achieving sufficient real-world accuracy is the handling of the inconsistency and quality of the acquired biosignal. For example, acquiring high quality PPG signals can be challenging because they are typically created under a diversity of conditions set forth by sensors (e.g., camera hardware, numerous flash settings), users (e.g., body weight, skin tone, ages), and environments (e.g., contact grips, lighting conditions). There is no canonical “ground truth” PPG, since even purpose-built PPG devices in controlled environments may produce different signals for the same subject by small changes in placement or orientation of the device.

Machine learning (ML) has been used to attempt to address the issues and complexity described above in high quality biosignal acquisition. Traditional ML methods such as a Support Vector Machine (SVM) algorithm or regression have shown promising results but require perhaps more than fifty or so features extracted by hand, even when using biosignals, such as PPG signals, from purpose-built devices in controlled environments. In recent years, neural networks have been used in different ways to successfully improve performance, especially with smartphone-sourced PPG for example. But what has been achieved in this way is still insufficient due to the large diversity of signals and expensive, limited training data. One common theme is that poor PPG signals are often thrown out during training and are not considered during inference, leading to many failed or inaccurate readings. Another common theme is that models may work well to acquire a biosignal only on a limited number of smartphones or other devices.

In some cases, other biosignals, such as a vocal signal for example, can be alternatively or additionally used to assess a patient's health risks and predict health events. Alternatively or additionally, patient demographics or other health information can also be used to assess a patient's health risks and predict health events. Patient demographics and other health information, such as from an electronic health record or input by a patient or provider, can be manually or automatically entered or can be determined from a measured biosignal, such as a PPG or vocal signal. Specific signal characteristics or features differ based on patient demographics. For example, a PPG signal from older individuals may have a less prominent dicrotic notch or a more gradual rise to the systolic peak. Further, the measured biosignals can validate various self-reported medical labels received from a patient and/or input by a provider. For example, a patient diagnosed with diabetes by a medical doctor can have their PPG analyzed to validate the diabetes diagnosis and perhaps provide additional information about the diagnosis. The additional information the PPG can provide could be assessed after comparing the patient's measured PPG signal signature to other patients with a similar or the same PPG signal signature and determining the disease condition of the similar or same patients. Such a comparison can give the patient information about how the disease will progress given the features or characteristics of the patient's unique PPG signal signature at the time of the measurement or over time if multiple measurements were taken. The features or characteristics that differ in the PPG signal in this example (or any biosignal) can include amplitude, smoothness or shape of the signal curve, frequency, and phase or any combination of characteristics.

Embodiments described herein address the problems discussed above and may achieve real-world accuracy with limited data. The embodiments involve real-time environment assessment and problematic issue detection, training an AI model to measure signal quality so as to select high-quality signals among signals having a range of qualities, and domain adaption and transfer learning to make use of publicly available datasets, just to name a few examples. Embodiments also address the problems of assessing a patient's physiological state and predicting various health conditions and/or health events the patient may or will experience with a particular prevalence assigned to each.

In particular, embodiments described herein are directed to systems, methods, and apparatuses for more conveniently and accurately measuring, assessing and predicting one or more health-related risks or events, including but not limited to blood pressure and heart rate, for example. In some embodiments, the health-related characteristics may include blood oxygen level, blood glucose level, hemoglobin A1C, heart rate variability, hemoglobin, respiration rate, blood oxygen, or arrhythmia.

One embodiment is directed to a method for determining one or more health-related quantities for a person based on video images captured using a mobile device (e.g., a smartphone). The method may include steps, stages, or operations that include activation or launch of an application hosted by the mobile device and collecting a sample or samples of video of a person's fingertip or tissue from another part of the body, such as a patient's earlobe. For example, during data collection, participants may have equipment placed on them, such as a pulse oximeter placed on the finger, toe, and/or earlobe, a single-lead Electrocardiogram (ECG), or an Inertial Measurement Unit (IMU) taped to the sternum. Collection of data from these sensors may include a participant's blood pressure, which may be taken using the auscultation method (e.g., using a stethoscope to listen for Korotkoff sounds).

The method may also include processing the collected sample(s) to approximate a PPG signal and detection of possible data collection problems during acquisition of user data, which can occur under normal use in diverse conditions. The method may further include evaluating quality of the PPG signal that may have been gathered from diverse subjects and cameras under diverse conditions and passing the PPG signal as input to a trained blood pressure AI model. As discussed above, additional biosignals can be measured to help assess health risks and predict health events of the patient.

The activation or launch of the application may involve the application setting collection parameters and equipment characteristics, such as device manufacturer and model, camera or video settings, lens, or video processing options, and so on. If needed, in some embodiments, the application may collect data regarding the patient, such as skin tone, and other data regarding local environment, lighting conditions, stability/motion of device, and so on, before beginning video capture. Collecting a sample or samples of video from a person's fingertip (or other tissue) using a mobile device may involve use of the application's user interface (UI) to guide a user in the placement of their fingertip and in holding the mobile device correctly and steadily to collect the data. A camera (and flash LED) of a mobile device, such as a smartphone, or other portable device may be used.

Processing the collected sample(s) to approximate a PPG signal may involve a PPG signal generator module or similar functionality that may continuously process raw camera frames by extracting average red (R), green (G), and blue (B) values of all the RGB pixels in each video frame. In some implementations, the red channel is taken to approximate the PPG signal. Obtained red channel signals may be smoothed using a Butterworth filter or other filter and up-sampling may be performed using linear interpolation, for example. A Butterworth filter is a type of signal processing filter designed to have a frequency response as flat as possible in the passband. It may also be referred to as a maximally flat magnitude filter.

In some implementations, data collection problems (problematic issues) during acquisition of user data may be detected. Such problems may occur under normal use in diverse conditions. In other words, issues or factors that could result in unreliable or unusable data may be detected. For example, ambient conditions or factors may include motion of camera (e.g., relative to finger), movement of finger (e.g., relative to camera), improper position of finger, and a change in lighting that impacts an ability to accurately and reliably extract a PPG-like signal from camera frames. An Issue Detector module or such functionality may be included in the system architecture, as described below.

Camera settings may be adjusted to get a “good” (e.g., acceptable) signal during diverse conditions (ambient lighting, skin tone of user, mobile device camera, etc.). In some implementations, previously measured or entered skin tone information of a user may be retrieved from memory of the user's mobile device. An output that includes a determined disease state or a biomarker may be modified based on the retrieved skin tone information. In some embodiments, to at least partially control ambient factors/conditions, an Environment Assessor module may step up the light sensitivity periodically (e.g., every 1.2 seconds), subsequently holding constant the light sensitivity when the red channel intensity, for example, from the camera frames reach a minimum (predetermined) threshold. The Environment Assessor module may also adjust the intensity of light emitted by a flash LED system of the camera.

Evaluating the quality of the PPG signal gathered from diverse subjects and cameras under diverse conditions may involve identifying individual pulses in the PPG signal. For example, in one embodiment, a Pulse Generation module or such functionality may identify a peak or valley that expresses an individual pulse in the PPG signal. A signal quality evaluation process may reject or remove from further processing pulses that fail to meet the threshold required for processing and evaluation. In one embodiment, a Signal Quality Assessor module may be a neural network trained to assess individual pulses. During training of the Signal Quality Assessor module, collected fingertip data (or similar type of data for other tissue or body part) may be manually annotated (described below) with an “acceptable” or “not acceptable” label to produce training data for a model. For example, an “acceptable” pulse is uniform (multiple pulses are consistent among themselves) and has the characteristics of a PPG signal (e.g., generally having a steep slope up to a relatively sharp peak and having zero or up to 2 smaller notches on the way down).

Based on environmental and signal quality conditions (during signal acquisition), and an absence of data acquisition problems, the PPG pulses may be passed as input to a trained AI blood pressure model. In one embodiment, the blood pressure model is a neural network that continuously takes in “acceptable”-quality pulses from the PPG signal as input and produces systolic blood pressure (BP) and diastolic BP as output. The model averages the produced systolic and diastolic BP values to produce final BP values that are returned to the user.

As mentioned above, techniques that may achieve real-world accuracy may involve training an AI model to measure signal quality so as to select high-quality signals from signals having a range of qualities. In one embodiment, an AI model training process may involve, due to limited training data, a base model that is trained on publicly available, synchronized, PPG and BP datasets. The trained base model may be used as a basis for continued training using learning on collected (e.g., historical) pulse oximeter PPG, smartphone fingertip video, and blood pressure data, as described below. Although examples described herein involve a fingertip, other tissue or parts of the body may be used instead of, or in addition to, the fingertip. Claimed subject matter is not limited in this respect. A smartphone-sourced PPG signal produced from fingertip videos may also be used by applying a Butterworth filter, and up-sampling the pulse oximeter PPG and smartphone-sourced PPG to match the frequency of the publicly available data using linear interpolation, for example.

Additional AI training data may be captured for use in training the base model, such as from users of the application, training data collected under controlled conditions, and continuous data acquisition and model training through data collection along with accurate measurement of health quantity using a separate device(s). When in use, data collection and processing may be performed on a user's device, wherein the user may maintain privacy.

Output from the trained AI model may provide information representing a measure of one or more of the person's blood pressure, heart rate, SpO2, risk for certain health conditions like hypertension, or other health-related quantity. Based on the model output, one or more tasks may be executed by an application hosted by a mobile device, such as providing results via an application programming interface (API) call back that enables a developer to use the data in their own application, showing trends in the measured quantities over time, tuning medication dosages based on changes in the measured quantities, placing the measured data into an electronic health record (EHR) for use by a physician, and statistically analyzing the measured data in conjunction with other data or information (such as sleep quality, stress, or anxiety levels, etc.). Other tasks that may be performed by a mobile device, for example, may include generating an alert to a user that a measured quantity exceeds a preset threshold value, wherein the average value of a measured quantity exceeds the preset value over a set time period, and wherein the measured value in combination with another aspect of the user (BMI, weight, blood glucose level, etc.) suggests a problem needing attention. Still other tasks may include generating a phone call to an emergency number if a measured quantity alone or over time exhibits a characteristic of serious medical problem (such as stroke, etc.) and generating a recommendation to engage in an activity, undertake a change in behavior, etc.

As described above, in various embodiments, biomarkers may be estimated from biosensor data, which may be an optical signal like a PPG, electrical signals like an ECG, and so on. The optical biosensor data may be acquired from a smartphone-captured video of a person's finger or from a PPG sensor in devices such as a pulse oximeter. For example, measurements of real-time blood pressure and heart rate data may be acquired via fingertip video obtained with a mobile device. In an implementation, the mobile device may include a data measurement module (DMM), which may comprise a software development kit (SDK) embedded into an operating system of the mobile device. A user may use the mobile device (e.g., a smartphone, wrist-worn wearable such as smartwatch or smart wristband, carbud, or tablet) to measure their blood pressure and heart rate, for example, by operating the DMM, which may navigate the user to a particular view (e.g., menu of options and/or instructions) on the smartphone. The user may place their fingertip against the lens of the mobile device camera, and after about a minute or so, for instance, the DMM may provide the user's blood pressure and heart rate data to an application on the mobile device or external to the mobile device. In some implementations, video frames and related data may be processed completely on the device so that user identities may be completely unknown outside the device, for anonymity.

In some embodiments, the DMM works by extracting the red channel from each frame of the fingertip video to form a PPG signal. As explained above, PPG is commonly used in methods for measuring blood volume changes and can be used to derive health measures like heart rate, SpO2, and blood pressure. A neural network may be used to extract these health measures from the smartphone signal produced by the DMM.

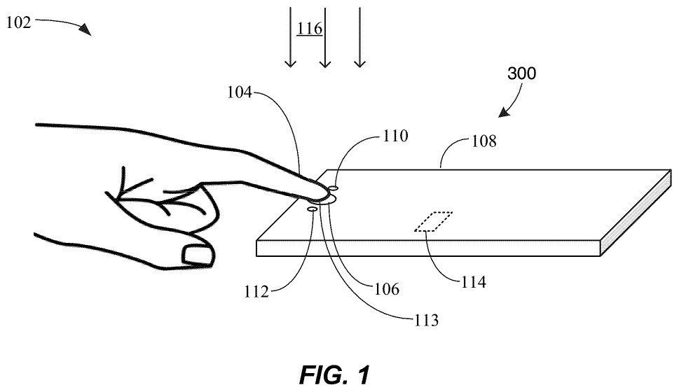

FIG. 1 illustrates a data gathering process 102 involving tissue of a fingertip 104 of a user engaging a camera 106 of a smartphone 108 for measuring a biomarker or other health-related quantity, or for determination of a health condition, according to some embodiments. In other example embodiments, other tissue or portion of a body may be used. Claimed subject matter is not limited to measurements based on a fingertip. In the situation illustrated, a camera lens on the back of smartphone 108 is used. Generally, fingertip 104 may be held substantially steady on or over camera 106 (e.g., or its lens) for several seconds to a minute or so while a biomarker application, for example, hosted by smartphone 108 causes the camera to capture images of the fingertip. Images may be captured at a frequency of milliseconds or seconds, depending on instructions from the biomarker application. For example, a frequency of image capture may be up to several dozen frames per second if the images are to be analyzed or processed as a video signal.

Conditions that exist during image capture may be important and thus considered during image or video processing, as explained below. Lighting conditions, motion conditions, and orientation of the smartphone may be important by affecting the quality of the images or the video that will subsequently be used to arrive at a pseudo PPG signal. Smartphone 108 may include an onboard flash LED 110, an onboard light sensor 112, a capacitive touch sensor 113, and an onboard accelerometer 114 (or IMU). Flash LED 110 may provide light to the fingertip and the surrounding area. This may light illuminate fingertip 104 with an intensity that allows for successful image capture by camera 106. The biomarker application hosted by the smartphone may at least partially control the intensity of light emitted by flash LED 110. Light sensor 112 may measure the intensity of ambient light 116 impinging on the light sensor. This intensity may be indicative of illumination of fingertip 104 and the surrounding area. Light sensor 112 may provide its measurements to the biomarker application hosted by the smartphone. In turn, the application may control the flash or exposure settings of the camera based on the light sensor measurements.

Capacitive touch sensor 113 may produce a signal based on whether a fingertip or other tissue is in contact with the camera (or a lens or lens cover thereof) and accelerometer 114 may measure motion and orientation of smartphone 108. Such motion may be vibratory motion or longer time-constant motion, for example. For successful image capture that can lead to high-quality pseudo PPG signals, relative motion between fingertip 104 and camera 106 should be small or non-existent. As described below, measurements by light sensor 112, capacitive touch sensor, and accelerometer 114 may be used by the biomarker application to assess the quality of captured images, video, or resulting pseudo PPG waveforms. For example, such measurements may be grouped as metadata for the captured images or video. Accordingly, the metadata can be used to determine the conditions that existed during acquisition of the images or video.

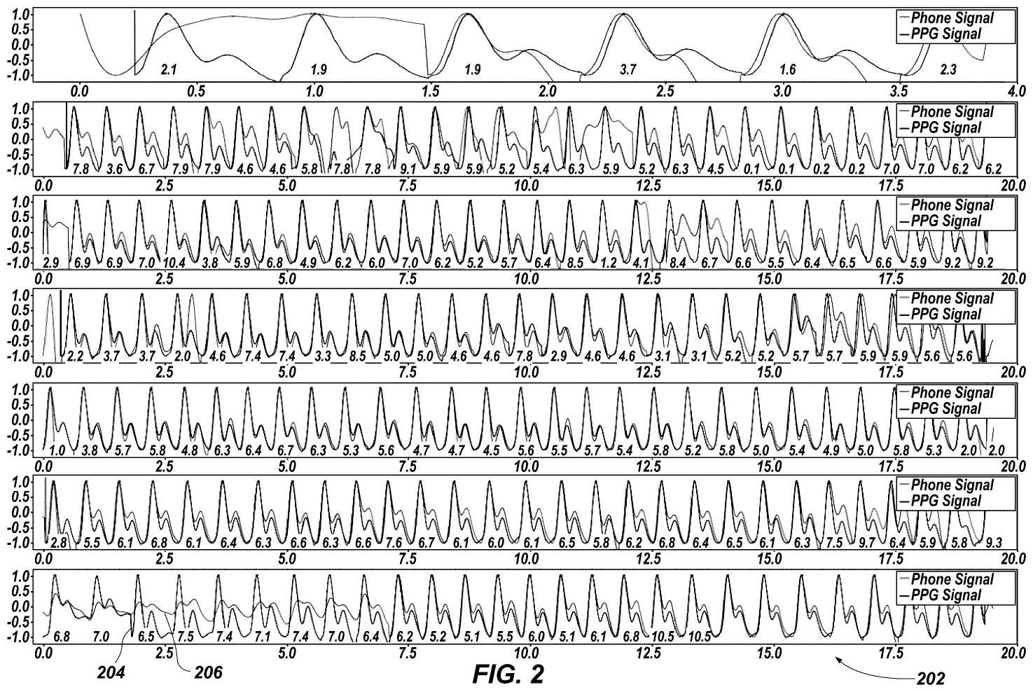

FIG. 2 illustrates example PPG waveforms 202 and illustrates the similarity between a PPG signal 204 obtained from a pulse oximeter and a red channel signal 206 derived from the red channel of a video signal captured by a smartphone (e.g., 108) or other similar portable device. As can been seen from the figure, the red channel signal 206, which corresponds with blood flow in the fingertip, is close to and mimics the behavior of PPG signal 204 when environment and signal quality are adequately controlled. This allows for the red channel signal being a suitable substitute for an actual PPG signal and provides a basis for which signal capture and signal quality techniques described herein are utilized.

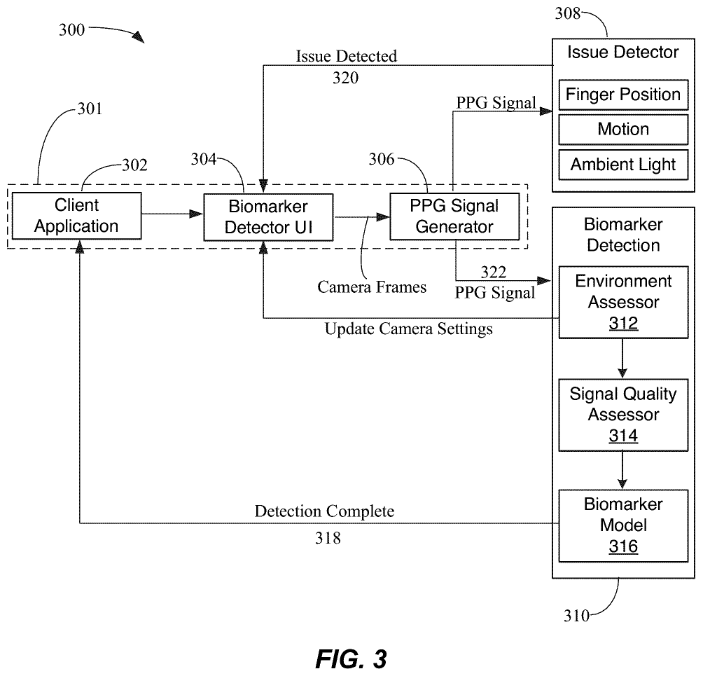

FIG. 3 is a block diagram of a system 300, illustrating a process flow and components that may be used in implementing an embodiment of the disclosed methods. For example, system 300 may be a DMM that includes sub-systems, elements, components, or modules that implement functionality for measuring blood pressure, among other biomarkers. Specifically, system 300 may include a client application 302, a Biomarker Detector user interface (Biomarker Detector UI) 304, a PPG Signal Generator 306, an Issue Detector 308, and a Biomarker Detection module 310. Client application 302 may be similar to or the same as the biomarker application described above.

Client application 302 may comprise computer-executable instructions that are installed on a user's device, which may be a mobile device such as a smartphone, wrist-worn wearable such as smartwatch or smart wristband, earbud, or tablet. In some embodiments, the application uses APIs to integrate functionality for measuring blood pressure or other health-related quantities with the capabilities of the mobile device. Client application 302 may use the APIs to navigate to a user interface associated with a video/data collection service and to provide blood pressure values when complete. In some embodiments, client application 302 may be developed using an SDK that is provided to enable access to, and use of, models and data processing methods, described below.

Biomarker Detector UI 304 is a user interface component that guides (e.g., provides real-time instructions for) the user in measuring their blood pressure or other biomarkers. Biomarker Detector UI 304 (and underlying services or functions) may provide tools to enable the user to initiate the video/data collection used to determine their blood pressure and/or provide a mechanism to interact with other features or functionality of system 300 and methods described herein. For example, the Biomarker Detector UI may display a view that provides instructions and shows feedback to the user, captures camera frames, and continuously or periodically passes the frames to PPG Signal Generator 306. For example, Biomarker Detector UI 304 may guide the user in placing their fingertip correctly on the camera, holding the smartphone correctly and steadily, and may provide the user with measurement progress and may indicate any errors in the process. Client application 302 may determine such measurement progress based, at least in part, on measurements received from accelerometer 114, for example. Biomarker Detector UI 304, via client application 302, may keep the camera flash LED on, and may control its intensity, to illuminate the finger during image capture.

Biomarker Detector UI 304 may be part of a service or application that interacts with Issue Detector 308 to control the acquisition of video/data and processing of the acquired video/data into a PPG signal by PPG Signal Generator 306. In some embodiments, Biomarker Detector UI 304 may be provided as a part of an SDK. In some embodiments, Biomarker Detector UI 304 may allow for measurements of a different health-related quantity (e.g., other than, or in addition to, blood pressure). PPG Signal Generator 306 may provide PPG signals to Issue Detector 308 and Biomarker Detection module 310.

A blood pressure detection process, performed by Biomarker Detection module 310, may begin once no issues (e.g., as determined by Issue Detector 308) have been identified during an initial time period, such as the first five seconds of the process, for example. A PPG signal may be passed through the following components to produce blood pressure: an Environment Assessor 312, a Signal Quality Assessor 314, and a Biomarker model 316. There generally may be many variables that impact the quality of the PPG signal that in turn impact the accuracy of Biomarker readings. Such variables may include ambient lighting conditions, skin tone, and smartphone camera quality. To control for this, Environment Assessor 312, via client application 302, may hold constant the light sensitivity and shutter speed of the camera (e.g., 106) of the mobile device at values that produce a minimum threshold of intensity. In some implementations, Environment Assessor 312 may step up the light sensitivity periodically, such as every 10 seconds, for example. In another implementation, Environment Assessor 312 may step up the light sensitivity by 75 ISO every 1.2 seconds, locking the light sensitivity when the red channel intensity from the camera frames reaches a minimum threshold. These values (e.g., 75 ISO and 1.2 s) may be determined empirically to increase the red channel intensity as fast as possible while stabilizing at each increment. Claimed subject matter is not limited to such values.

An updated PPG signal with camera settings (e.g., as metadata, such as time stamp of image capture, exposure time, aperture, resolution, and so on) are passed through Signal Quality Assessor 314, which may produce individual pulses from signals passed from Environment Assessor 312 and may pass acceptable pulses to Biomarker model 316. In one embodiment, Signal Quality Assessor 314 is a neural network trained to assess individual pulses. Training data may be manually annotated based on collected user fingertip data with a label of “acceptable” or “not acceptable” to produce training data for this model. In general, an “acceptable” pulse is uniform (consistent with other pulses) and has the characteristics of a PPG signal (generally a steep slope up to a relatively sharp peak and has 0 to 3 smaller notches on the way down), though claimed subject matter is not so limited.

Biomarker model 316, which is a neural network, may depend on relatively high quality signals to produce a good result. In some implementations, a time-series of image frames of video are partitioned into video chunks (e.g., segments) that each have a predetermined time span. Accordingly, Signal Quality Assessor 314 looks at such chunks of signals provided by Environment Assessor 312 and only passes acceptable signals to Biomarker model 316. For example, such chunks of signals may be two-second long segments of a PPG signal. In some implementations, two contiguous video chunks may be combined into a video segment having a portion that comprises an overlap between the two contiguous video chunks. Signal Quality Assessor 314 may generate a quality score determined by comparing a signal quality feature of a PPG signal provided to Biomarker Detection 310 to a same-type signal quality feature of a deep learning-based model PPG signal, which may be stored in computer memory or updated and provided by Biomarker Model 316, for example. In other implementations, Signal Quality Assessor 314 may generate a quality score based on metadata included in the PPG signal provided to Biomarker Detection 310.

In some implementations, Signal Quality Assessor 314 is a neural network trained to assess chunks of PPG signals. Biomarker Model 316 may produce a systolic BP and diastolic BP as output. The model may average the produced systolic and diastolic BP values to produce final BP values that are returned to the user. After acceptable PPG/red channel signals have been processed, the blood pressure values may be provided to the user through client application 302. In some embodiments, the blood pressure values, peak values, changes over time, and so on may be used as input data to a set of rules or a trained model to determine whether to generate an alert, place a call to a medical provider, recommend an activity or change in behavior to the user, etc. In some embodiments, Biomarker model 316 is based, at least in part, on a neural network that is trained using transfer learning on pulse oximeter PPG data and information collected or produced by a mobile device hosting client application 302, as described below. Such information may include video chunks or blood pressure data previously determined by the mobile device, for example.

In some embodiments, as mentioned above, fingertip data, such as in the form of PPG segments, may be annotated (automatically or manually) as being “acceptable” or “not acceptable” to produce training data for Biomarker model 316, which is a neural network that continuously or periodically takes in as input, “acceptable”-quality, segments (e.g., two-second long chunks) of the PPG signal and produces systolic BP and diastolic BP as output. Biomarker model 316 may average the produced systolic and diastolic BP values to produce final BP values that are returned to the user. In some implementations, the training process for the Biomarker model 316 may be performed by first training a base model on publicly available, synchronized, PPG and BP datasets. The trained base model may continue to be trained using transfer learning on pulse oximeter PPG, smartphone fingertip video, and blood pressure data subsequently collected. The smartphone PPG may be produced from fingertip videos in a similar process on the mobile device. For example, this process may include using red channel data of a PPG, applying a Butterworth filter, and up-sampling pulse oximeter PPG and smartphone PPG to match the frequency of the publicly available data using linear interpolation.

PPG Signal Generator 306 may receive video frames collected by a camera in the user's device and may perform the signal/image processing to generate the PPG/red channel signal for further analysis and evaluation. For example, PPG Signal Generator 306 may continuously or periodically process raw image frames by extracting the average red, green, and blue pixel values across an array of pixels in each frame. In some implementations, a single image frame at a time is processed. In other implementations, two or more image frames are processed at a time. The red values may be smoothed with a Butterworth filter and up-sampled via linear interpolation for Biomarker model 316. Such processed frames and associated PPG signal may be passed to the UI of client application 302 as “detection complete,” as indicated by arrow 318.

Issue Detector 308 may be responsible for implementing a set of processes to determine if the video/data collection should be interrupted, stopped, or discarded due to unreliability caused by one or more of the user's finger position, motion of the camera or user's finger, or ambient light conditions, among other possible sources of error. Patient physiological data, in the form of a PPG signal, for example, may include metadata, which may be measured by an IMU of the camera, or other elements that may be used as signal quality features. The patient physiological data, which may also include a disease state or a biomarker feature, may be provided to Issue Detector 308 from PPG Signal Generator 306, for example. Signal quality features and disease state or biomarker features may be manifested as characteristics or parameters of the PPG signal, for example. For example, a PPG signal having such characteristics and parameters may be provided to Issue Detector 308 to determine signal quality (e.g., a quality score) or whether problematic issues occurred during the measurement process that led to the PPG signal. The PPG signal having such characteristics and parameters may also be provided to Biomarker Detection 310 to determine a disease state or biomarker. For example, a process for determining the disease state or biomarker of patient physiological data based on a disease state or a biomarker feature and a quality score may include determining that the quality score exceeds a threshold acceptance value and determining the disease state or the biomarker of the patient physiological data based on the disease state or biomarker feature.

Issue Detector 308 may use such metadata to determine ambient or environmental conditions that existed during the measurement process that produced the patient physiological data. For example, metadata measured by an IMU (e.g., or an accelerometer) of the camera may include relative motion between the camera and a user's fingertip and the orientation of the camera (e.g., and thus the orientation of the mobile device housing the camera).

In some embodiments, translational and rotational motion of the camera may be represented by one or more motion signatures, which may be represented by a waveform or plot of values. For example, translational motion may be a plot or table of time-dependent values of displacement of the camera relative to a fixed point in space. The plot or table may lead to a motion signature for the camera during a time span. Similarly, rotational motion may be one or more plots or tables of time-dependent values of pitch, yaw, and roll of the camera relative to a fixed axis. The plots or tables may lead to other types of motion signatures for the camera during a time span. Various features of the motion signatures may be analyzed. Such features may include frequency-of-motion (e.g., Fourier analysis of motion signatures in the frequency domain), shapes of waveforms or curves, and duration of such features (e.g., when they occurred relative to the time of data acquisition and for how long they occurred), just to name a few possible features. For example, a motion signature expressing displacement of the camera may include a feature that indicates that the camera was not resting on a stable surface (e.g., a tabletop). Such a feature may be a relatively low frequency (e.g., 5-10 Hz) component of the motion signature, which may be a result of the camera being hand-held during data acquisition. This type of motion may likely adversely affect the quality of data acquired during the time of this motion because the proximity of the fingertip to the camera may be difficult to hold constant with this type of motion. In another example, a motion signature expressing angular displacement, such as a relatively slow roll, of the camera may include a feature that also indicates that the camera was not resting on a stable surface. This type of motion, however, may not affect the quality of data acquired during the time of this motion because the proximity of the fingertip to the camera may nevertheless be held constant with this type of motion. Such dynamics of camera motion, as given in these and other examples, may be captured in motion signatures. Accordingly, analyzing these dynamics and how they affect motion of a fingertip or other tissue during data acquisition may allow for assessing the quality of the data.

In other embodiments, relative motion between the fingertip and the camera may be determined based, at least in part, on intensity measurements of pixel data of multiple image frames. For example, substantial changes of light intensity among image frames may indicate to Issue Detector 308 that relative motion occurred during capture of these image frames. In still other embodiments, relative motion between the fingertip and the camera may be determined based, at least in part, on motion signatures in combination with intensity measurements of pixel data of multiple image frames. In still other embodiments, relative motion between the fingertip and the camera may be determined to be zero if a signal from a capacitive touch sensor (e.g., 113), which may be onboard the handheld device and relatively near or on the lens of the camera, indicates continuous contact between the fingertip and the camera during data acquisition.

If an issue is detected, then Issue Detector 308 may send a signal or message to Biomarker Detector UI 304, as indicated by arrow 320, to discard or prevent the further processing of particular samples of the collected frames of video/data. Moreover, Issue Detector 308 may send a signal or message to Biomarker Detector UI 304 including instructions for the Biomarker Detector UI to adjust in real-time a parameter of ambient conditions, the adjusting being based on the determination, by the Issue Detector, that the signal characteristic of the pseudo PPG signal does not meet a quality criterion. The ambient conditions may include relative motion between the camera and the user's finger and the ambient light to which the sensor is exposed. Such adjustments in real-time may occur during the measurement process.

In particular, Issue Detector 308 may continuously or periodically look for issues that prevent production of high-quality signals. System 300 may include a detector, or process of detection, for each problematic issue, which, upon detection, may be provided as feedback to the user, via the UI of client application 302. For example, if a detector detects a problematic issue, the user may be alerted and Biomarker Detection module 310 may be reset.

As mentioned above, some examples of problematic issues may include a finger-off condition, motion/unstable ambient lighting condition, and phone orientation problem, just to name a few examples. In a particular implementation, a finger-off condition may be detected by gathering the last 500 milliseconds (ms) of video frames and analyzing the ratios of red to blue and green to identify whether the user's finger is correctly on the camera. A threshold for the ratio values may be dynamically selected based on the overall intensity in the collected frames to account for variables such as skin tone and camera settings. These thresholds may be identified empirically through collected fingertip video data where fingers are determined to be on or off at different intensities and camera settings. The image frame may also be segmented into quadrants (e.g., or other proportions), with ratios and thresholds being determined for each quadrant, with finger-off condition being identified if at least one quadrant's ratio is below a threshold. In another implementation, a finger-off condition may be detected by analyzing motion signatures of the camera, as described above.

In a particular implementation, a motion/unstable ambient lighting condition may be detected by gathering the last 500 ms of video frames and looking for the difference between minimum and maximum red pixel values. If the difference is more than a threshold value (e.g., 20 in a range of 0-255), it may be determined that too much motion has occurred or that the ambient lighting is too unstable. A phone orientation problem may occur if the user does not hold the phone relatively flat, thus affecting the amount of ambient light that reaches the camera. An on-board accelerator in the phone may be used to determine if the phone is flat, for example.

Assuming that no problematic issues, such as those described above, are detected, PPG Signal Generator 306 may generate a PPG signal/red channel, as indicated by arrow 322, for further analysis and evaluation by Biomarker Detection module 310.

In some embodiments, the services and functionality of system 300 may be combined with different ground truth data to generate measures of other health-related quantities and/or to provide greater insight into a user's health. For example, system 300 may be used to provide one or more of the following measurements if the appropriate ground truth is available: SpO2, involving use of a hypoxia tent to lower the subject's blood oxygen level and use of the SpO2 readings from a pulse oximeter; Blood glucose, involving the taking of blood glucose measurements via a finger stick and capturing data from a user before and after a meal; Hemoglobin A1C (HbA1c), involving taking a HbA1c measurement via a finger stick and lab analysis or point-of-care HbA1c analyzer; Heart rate variability, using an ECG device; Hemoglobin, using a hemoglobin measurement device; Respiration rate, by attaching a respiration rate band onto the user; and Arrhythmia, using an ECG device. Issue Detector 308, Environment Assessor 312, and Signal Quality Assessor 314 described above as applied to blood pressure and heart rate measurements may instead or also be used to make measurements of SpO2, blood glucose, heart rate variability, hemoglobin, respiration rate, and arrhythmia. Further, in some embodiments, the camera (image/video sensor) of the mobile device may be combined with other sensors on the device to collect additional data and signals that may be used to assist in evaluating a user's health or condition.

As discussed above, biomarkers, such as blood pressure, HbA1c, hypertension risk, and heart rate, for example, may be estimated based on biosensor data, such as a PPG. Optical biosensor data may be acquired from a smartphone-captured video of a person's finger using a system such as 300. For example, measurements of real-time blood pressure and heart rate data, or other biomarker, may be acquired via fingertip video obtained with a mobile device. In this context, for example, “real-time” refers to a situation where such biomarkers may be provided to a user while the user continues to be measured for the biomarkers, the latter measurements perhaps providing updated measurements. In an implementation, a user may use a smartphone, including a DMM, to measure their blood pressure and heart rate by placing their fingertip against the lens of the smartphone camera.

In some embodiments, a PPG signal may be acquired from a smartphone by averaging all pixels from the red channel of video of a fingertip from the smartphone. This process may produce a 1D (one-dimensional) time-series signal that mimics a PPG signal. However, this signal may generally be noisier than a signal acquired from a specially designed device like a pulse oximeter and may generally have a lower sampling rate. In one implementation, a region-of-interest (e.g., the middle 200×200 pixels) may be used. In another implementation, the frames of the smartphone video may be used to create the 1D time-series PPG signal using a deep learning model. Herein, a deep learning model refers to a neural network model that utilizes one or more of the following types of layers: convolutional, residual, recurrent, attentional, and transformer. For models operating on the smartphone video frames, there may be two different types of deep learning models used: a 2D model that operates independently on each individual frame, and a 3D model that operates on two or more frames at one time. In both cases, the model produces a time-series of embeddings that may be used to produce an output, which may be another embedding, a prediction of a pulse oximeter signal, a biomarker estimation, and so on.

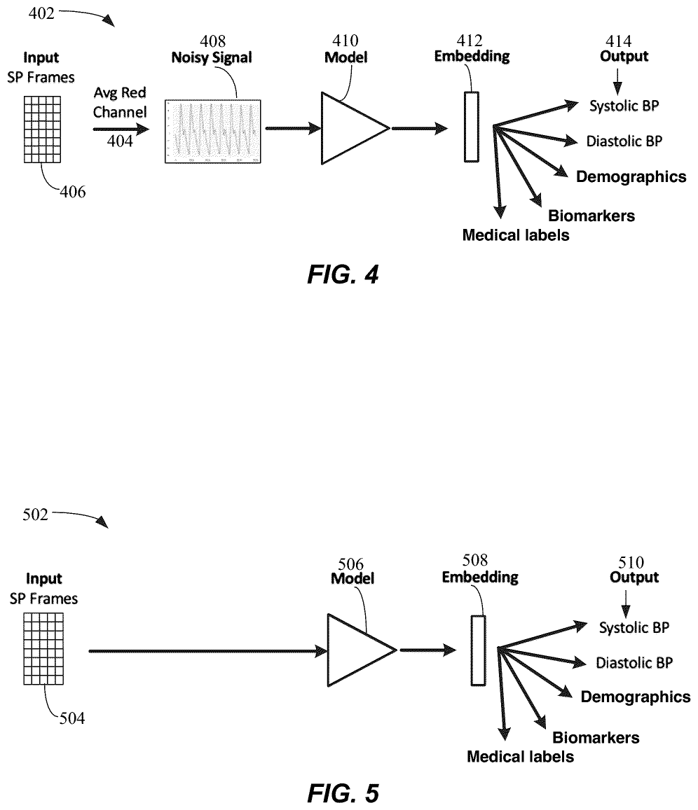

In one type of deep learning model, a baseline may be produced by a baseline biomarker estimation system that involves passing the averaged red channel signal or time-series of video frames through a deep learning model to predict the biomarker. FIG. 4 illustrates a process 402 that uses this type of baseline model, wherein the averaged red channel 404 of input video frames 406 is used to create a 1D time-series noisy signal 408 that resembles a PPG signal. This signal may then be passed through a deep learning model 410 that uses embedding 412 to produce an output 414, which may be predicted biomarkers such as systolic and diastolic blood pressure, demographics, other biomarkers, and various medical labels like health diagnoses and laboratory results values.

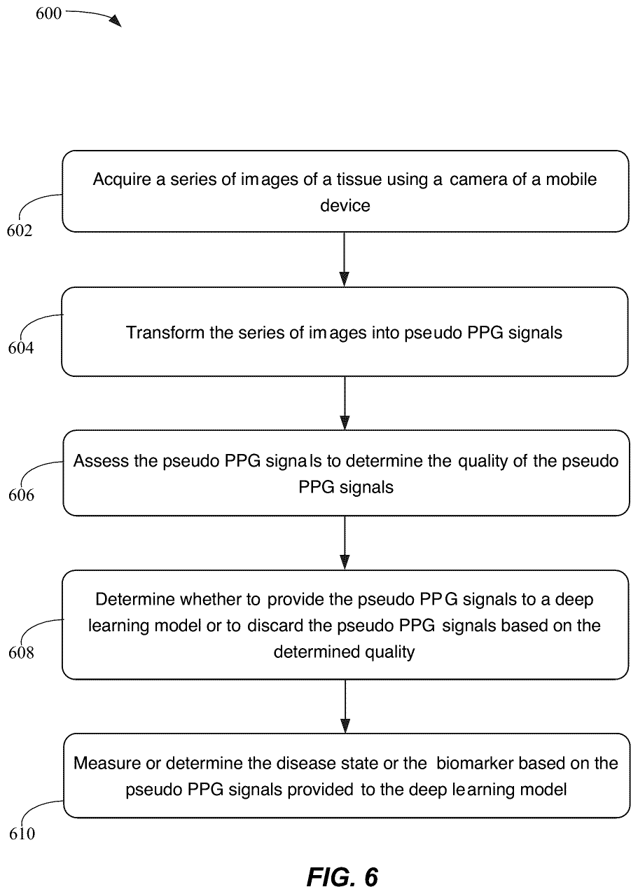

FIG. 5 illustrates another process that uses a different type of baseline model, wherein a time-series of video frames are passed directly through a deep learning model to predict the biomarker. Process 502 provides a time-series of video frames 504 directly to a deep learning model 506, which may use embedding 508 to produce an output 510, which may be predicted biomarkers such as systolic and diastolic blood pressure, demographics, other biomarkers, and various medical labels like health diagnoses and laboratory results.

In some embodiments, a deep learning model may be trained to use smartphone data to predict a paired pulse oximeter signal. The input to the model may be the 1D time-series smartphone data, described above, from the averaging of the red channel or computed from the frames of the video. The predicted pulse oximeter signal may then be used as input to the deep learning model that will predict the biomarker.

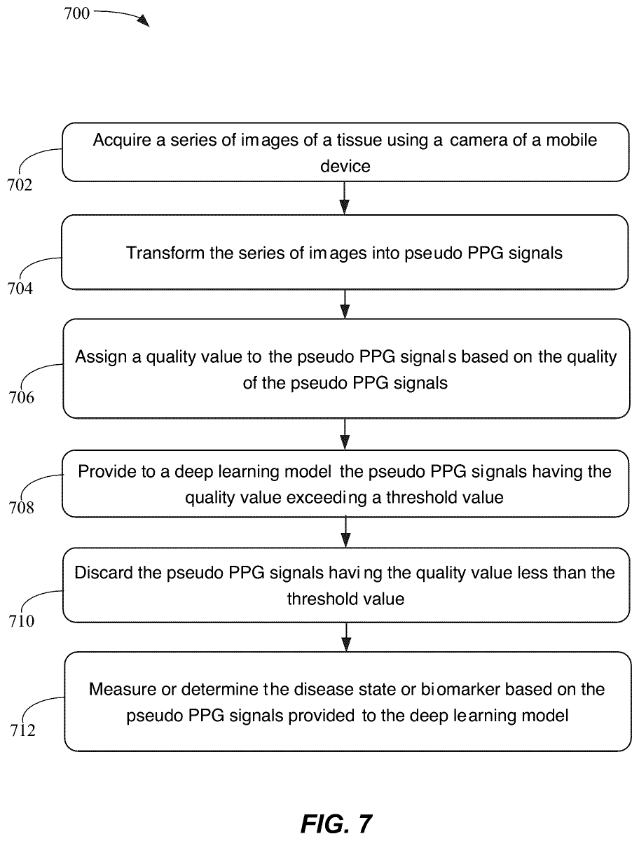

FIG. 6 is a flow diagram of a process 600 for addressing signal quality of a PPG signal, according to some embodiments. For example, a computer processing system (e.g., a processor) may perform process 600 that includes data gathering process 102 described above. At 602, the processor may acquire a series of images of tissue 104 using camera 106 of a mobile device (e.g., smartphone 108). For example, client application 302 may use Biomarker Detector UI 304 to operate the camera of a mobile device.

At 604, the processor may transform the series of images into pseudo PPG signals, as described above. At 606, the processor may assess the pseudo PPG signals to determine the quality of the pseudo PPG signals. The pseudo PPG signal may have a signal characteristic associated with a quality indicator of the pseudo PPG signal. In other words, a feature of the pseudo PPG signal, such as the detailed shape of a waveform or pulse, for example, may allow for quantifying or characterizing the quality of the pseudo PPG signal. The feature may also allow for determining the circumstances (e.g., ambient conditions) that existed during acquisition of the patient physiological data. Such a feature may be compared to, or used in conjunction with, metadata gathered from measurements of light sensor 112 and accelerometer 114 during acquisition.

At 608, the processor may determine whether to provide the pseudo PPG signals to a deep learning model or to discard the pseudo PPG signals based on the determined quality. The processor may compare the signal characteristic of the pseudo PPG signal to a characteristic or parameter of a signal characteristic of a deep learning-based model PPG signal. For example, after being provided to Biomarker detection module 310, the signal characteristic of the pseudo PPG signal may be compared to a characteristic or parameter of an analogous signal characteristic of a deep learning-based model PPG signal.

At 610, the processor may measure or determine the disease state or the biomarker based on the pseudo PPG signals provided to the deep learning model, as described above.

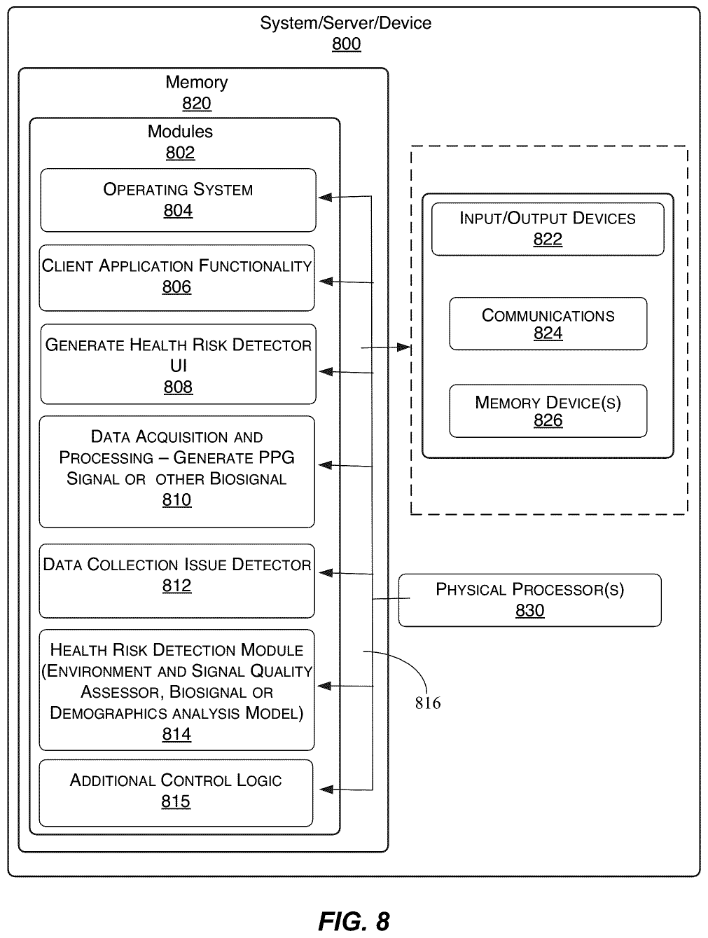

FIG. 7 is a flow diagram of a process 700 for determining signal quality of a PPG signal and determining a disease state or biomarker based on the PPG signal, according to some embodiments. For example, a computer processing system (e.g., a processor) may perform process 700 that includes data gathering process 102 described above. At 702, the processor may acquire a series of images of tissue 104 using camera 106 of a mobile device (e.g., smartphone 108). For example, client application 302 may use Biomarker Detector UI 304 to operate the camera of a mobile device. At 704, the processor may transform the series of images into pseudo PPG signals, as described above. At 706, the processor may assign a quality value to the pseudo PPG signals based on the quality of the pseudo PPG signals. In some implementations, the quality value may be a “pass” or “no pass” designation. In other implementations, the quality value may be a number (or a rating based on a number, such as lettering or other characters). The quality of the pseudo PPG signals may be determined by techniques described above.

At 708, the processor may provide to a deep learning model the pseudo PPG signals having the quality value exceeding a threshold value. In contrast, at 710, the processor may discard the pseudo PPG signals having the quality value less than the threshold value. In other words, steps 708 and 710 sort out the pseudo PPG signals according to their quality. If the quality is good, the pseudo PPG signals are used, but if their quality is bad, they are discarded. At 712, the processor may measure or determine the disease state or biomarker based on the pseudo PPG signals provided to the deep learning model.

In some implementations, in addition to measuring or determining a disease state or biomarker, an output may include displaying trends of the determined disease state or the biomarker over time. In some implementations, an application (e.g., 302) may modify a medication dosage based on the trends of the determined disease state or the biomarker. The application may then display to a user the modified medication dosage. In some cases, the application may analyze the determined disease state or the biomarker in conjunction with data based on sleep quality, stress, or anxiety levels of a patient. The display or other portion of a UI may further include an alert to a user that the determined disease state or the biomarker exceeds a preset threshold value. The display may also include a recommendation to engage in an activity or to undertake a change in behavior, and the real-time blood pressure and heart rate data.

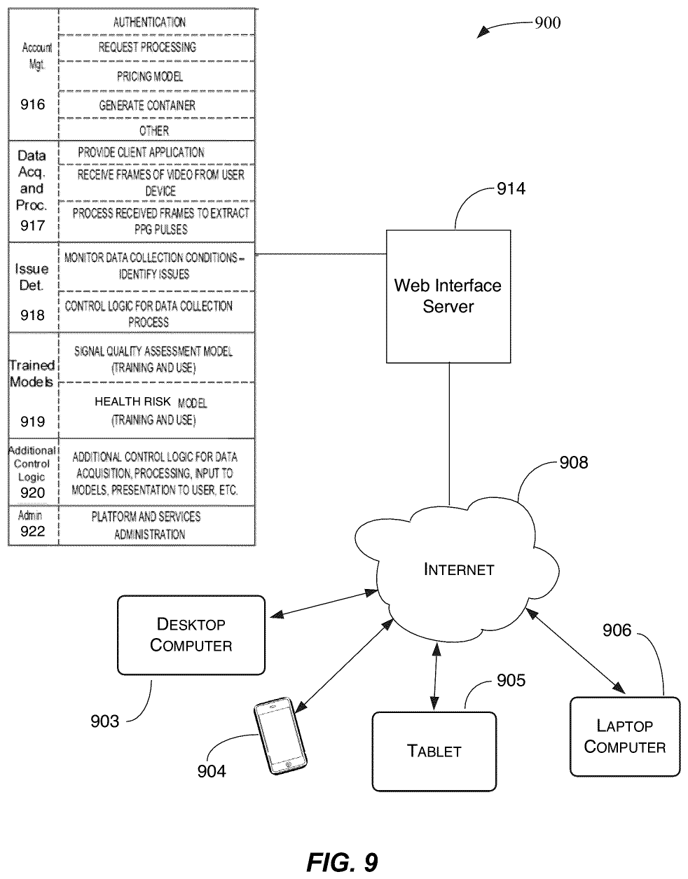

FIG. 8 illustrates elements or components of a computer device or system configured to implement a method, process, function, or operation of some embodiments of a system (e.g., 300) and methods described herein. As noted, in some embodiments, the system and methods may be implemented in the form of an apparatus that includes a processing element and a set of executable instructions. The executable instructions may be part of a software application and arranged into a software architecture.

In general, an embodiment may be implemented using a set of software instructions that are designed to be executed by a suitably programmed processing element (such as a GPU, CPU, microprocessor, processor, controller, computing device, etc.). In a complex application or system such instructions are typically arranged into “modules” with each such module typically performing a specific task, process, function, or operation. The entire set of modules may be controlled or coordinated in their operation by an operating system (OS) or other form of organizational platform.

Each application module or sub-module may correspond to a particular function, method, process, or operation that is implemented by execution of the instructions contained in the module or sub-module. Such function, method, process, or operation may include those used to implement one or more aspects, techniques, components, capabilities, steps, or stages of the described system and methods. In some embodiments, a subset of the computer-executable instructions contained in one module may be implemented by a processor in a first apparatus and a second and different subset of the instructions may be implemented by a processor in a second and different apparatus. This may happen, for example, where a process or function is implemented by steps that occur in both a client device and a remote server.

The application modules and/or sub-modules may include any suitable computer executable code or set of instructions (e.g., as would be executed by a suitably programmed processor, microprocessor, or CPU), such as computer-executable code corresponding to a programming language. For example, programming language source code may be compiled into computer-executable code. Alternatively, or in addition, the programming language may be an interpreted programming language such as a scripting language.

The modules may contain one or more sets of instructions for performing a method or function described with reference to the figures. These modules may include those illustrated but may also include a greater number or fewer number than those illustrated. As mentioned, each module may contain a set of computer-executable instructions. The set of instructions may be executed by a programmed processor contained in a server, client device, network element, system, platform, or other component.

A module may contain computer-executable instructions that are executed by a processor contained in more than one of a server, client device, network element, system, platform or other component. Thus, in some embodiments, a plurality of electronic processors, with each being part of a separate device, server, platform, or system may be responsible for executing all or a portion of the instructions contained in a specific module. Thus, although FIG. 8 illustrates a set of modules which taken together perform multiple functions or operations, these functions or operations may be performed by different devices or system elements, with certain of the modules (or instructions contained in those modules) being associated with those devices or system elements.

As illustrated in FIG. 8, system 800 may represent a server or other form of computing or data processing system, platform, or device. Modules 802 each contain a set of computer executable instructions, where when the set of instructions is executed by a suitable electronic processor or processors (such as that indicated in the figure by “Physical Processor(s) 830”), system (or server, platform, or device) 800 operates to perform a specific process, operation, function, or method. Modules 802 are stored in a memory 820, which typically includes an Operating System module 804 that contains instructions used (among other functions) to access and control the execution of the instructions contained in other modules. The modules 802 stored in memory 820 are accessed for purposes of transferring data and executing instructions by use of a “bus” or communications line 816, which also serves to permit processor(s) 830 to communicate with the modules for purposes of accessing and executing a set of instructions. Bus or communications line 816 also permits processor(s) 830 to interact with other elements of system 800, such as input or output devices 822, communications elements 824 for exchanging data and information with devices external to system 800, and additional memory devices 826.

For example, Module 806 may contain computer-executable instructions which when executed by a programmed processor cause the processor or a device containing the processor to perform the functions of the client application to enable the collection and processing of the acquired data either on the device or (as an example) to navigate to a server, upload the video or other data to the server, receive the output(s) of the blood pressure model from the server, and present the output(s) to the user (in the form of a measure, graph, chart, range etc.). Module 808 may contain computer-executable instructions which when executed by a programmed processor cause the processor or a device containing the processor to generate a health risk detector user interface for presentation to a user. The interface may provide the user with tools and instructions for collecting the video data using their device (such as a smartphone).

The functionality for data acquisition, data processing, issue detection, and the determination of blood pressure or another health-related quantity may be provided to a user in one or more formats. These include an SDK that developers can use to perform one or more of the functions (such as data collection, initial data processing, issue detection, running the blood pressure model) in their own applications. In another embodiment, a downloaded client application is provided that is capable of performing one or more of the functions directly to an end user. In another embodiment, certain functions of the client application may be replaced by services accessible through an account on a SaaS platform.

In any of these embodiments, the trained models may reside as services on a remote server or platform so that the models can be updated and continually trained and improved as new data becomes available. This arrangement may allow anonymized data collected from multiple users to be made available to other developers and model builders for use in determining other health-related quantities and for determining correlations between medical conditions, health, and the quantities. In any of these embodiments, the interface may be provided through the web, mobile, or desktop, as long as there is an attached video camera.

Module 810 may contain computer-executable instructions which when executed by a programmed processor cause the processor or a device containing the processor to acquire the video data and subject it to processing (image and/or signal processing) to extract the red channel of the video, and to treat this signal as a pseudo-PPG signal or other biosignal. Module 812 may contain computer executable instructions which when executed by a programmed processor cause the processor or a device containing the processor to monitor for issues or concerns that may occur during the video/data collection and to assist in determining if the collected video/data is useable for generating the pseudo-PPG signal or other biosignal. Module 814 may contain computer-executable instructions which when executed by a programmed processor cause the processor or a device containing the processor to perform functions used to generate a health risk detection measure from the acquired video in cases where the Issue Detector has not prevented further processing of the video frames. As illustrated in the figure, this may include consideration of environmental conditions and signal quality prior to inputting the pseudo-PPG signal (e.g., the red channel of the video) into a trained blood pressure model. Module 815 may contain computer-executable instructions which when executed by a programmed processor cause the processor or a device containing the processor to execute additional control logic for managing the decision processes involved in obtaining device or camera parameters, setting parameters of the video/data collection process, and controlling the video/data processing flow, configuring a response to the determined blood pressure measures, etc.

In some embodiments, certain of the functionality and services provided by the system and methods described herein may be made available to multiple users by accessing an account maintained by a server or service platform. Such a server or service platform may be termed a form of Software-as-a-Service (SaaS).

It should be understood that the embodiments described above can be implemented in the form of control logic using computer software in a modular or integrated manner. Based on the disclosure and teachings provided herein, a person of ordinary skill in the art will know and appreciate other ways and/or methods to implement the embodiments using hardware and a combination of hardware and software.

In some embodiments, certain of the methods, models or functions described herein may be embodied in the form of a trained neural network, where the network is implemented by the execution of a set of computer-executable instructions or representation of a data structure. The instructions may be stored in (or on) a non-transitory computer-readable medium and executed by a programmed processor or processing element. The set of instructions may be conveyed to a user through a transfer of instructions or an application that executes a set of instructions (such as over a network, e.g., the Internet). The set of instructions or an application may be utilized by an end-user through access to a SaaS platform or a service provided through such a platform. A trained neural network, trained machine learning model, or other form of decision or classification process may be used to implement one or more of the methods, functions, processes, or operations described herein. Note that a neural network or deep learning model may be characterized in the form of a data structure in which are stored data representing a set of layers containing nodes, and connections between nodes in different layers are created (or formed) that operate on an input to provide a decision or value as an output.

In general terms, a neural network may be viewed as a system of interconnected artificial “neurons” that exchange messages between each other. The connections have numeric weights that are “tuned” during a training process, so that a properly trained network will respond correctly when presented with an image or pattern to recognize (for example). In this characterization, the network consists of multiple layers of feature-detecting “neurons;” each layer has neurons that respond to different combinations of inputs from the previous layers. Training of a network is performed using a “labeled” dataset of inputs in a wide assortment of representative input patterns that are associated with their intended output response. Training uses general-purpose methods to iteratively determine the weights for intermediate and final feature neurons. In terms of a computational model, each neuron calculates the dot product of inputs and weights, adds the bias, and applies a non-linear trigger or activation function (for example, using a sigmoid response function).

Machine learning (ML) is being used more frequently to enable the analysis of data and assist in making decisions in multiple industries. In order to benefit from using machine learning, a machine learning algorithm is applied to a set of training data and labels to generate a “model” which represents what the application of the algorithm has “learned” from the training data. Each element (or example, in the form of one or more parameters, variables, characteristics or “features”) of the set of training data is associated with a label or annotation that defines how the element should be classified by the trained model. A machine learning model is a set of layers of connected neurons that operate to make a decision (such as a classification) regarding a sample of input data. When trained (i.e., the weights connecting neurons have converged and become stable or within an acceptable amount of variation), the model will operate on a new element of input data to generate the correct label or classification as an output.

An embodiment, which is provided as a particular example and is not intended to exclude other suitable methods, includes a methodology for training a biomarker estimation deep learning model, which may be trained on a large bank of biosignals (e.g., collected from public and private datasets). This may include PPG, vocal signals, ECG, seismocardiogram (SCG), ballistocardiogram (BCG), Electroencephalogram (EEG), and more. The output of the model may be a series of embeddings that are utilized to predict some downstream task, which is discussed further below.

The training may utilize both unsupervised and semi-supervised learning. For unsupervised, portions of the signal may be removed from the input sequence, and the model may be instructed to predict the missing portion of embeddings. In semi-supervised, certain characteristics of the signal may be calculated (such as heart rate from the PPG signal), for which the model will attempt to predict these portions. In another iteration of semi-supervised, certain characteristics about samples that are similar will be calculated (e.g., from the same person, have the same heart rate, etc.) and the model will be tasked with creating an embedding that is similar for these samples with the same characteristics.

The embeddings created by the model may then be used for biomarker estimation. A deep learning model may operate on the embeddings created by the large pre-trained model to predict the biomarker, which may be systolic BP and diastolic BP, for example.

For the large pretrained model, one may quantize the different values of the input signal. For example, for a signal that can take values of 0 to 1, one may create 1 million equally spaced states, where each value of the signal is rounded to the nearest state.

As discussed above, there may be multiple sources of time-synchronized sensor data collected at one time. For example, sources may include the pulse oximeter, IMU, ECG, or the smartphone optical and motion signals. These signals may be combined as separate channels of the input signal (e.g., channel 0=PPG, channel 1=accelerometer λ, channel 2=accelerometer Y). The deep learning model would then operate on this multi-channel signal.

The deep learning model may be used to predict not only the biomarker of interest, but also additional features about the signal. For example, heart rate may be calculated from the PPG signal. The deep learning model would be instructed to predict both heart rate and the biomarker of interest. The prediction of these additional features can improve the performance of the biomarker prediction.

One such example is the prediction of pulse transit time (PTT). Using an IMU signal (motion) and optical signal, the time interval from when the aortic opening is sensed by the IMU and a landmark point in the optical signal (e.g., peak, valley, beginning of rise to systolic peak) may be measured. This time interval is PTT, which may be predicted by a model that contains both IMU and optical data. One may train different machine learning models for different demographics. For example, one may train a model for individuals of ages 0-40, 40-60, and 60+. During inference, one would use the model that matches the individual's demographics.

The data collected from the car and toe pulse oximeters may be used to calculate the PTT. By using a landmark point in both signals (e.g., peak, valley, location of rise to systolic peak, etc.), the time interval between the landmark point in the car and toe signals is calculated. Next, a deep learning model running on either just the optical fingertip signal or optical fingertip and IMU data may be used to predict that PTT value.

An embodiment, which is provided as a particular example and is not intended to exclude other suitable methods, includes a methodology for collecting training data for a model or models. A data collection process may include, for example, a data collection person or persons. They first guide a subject through a relaxation period, such as about five minutes, which may allow for stabilizing the subject's blood pressure. Then they may take blood pressure readings (discarding the first) and alternative blood pressure readings and fingertip videos on a variety of smartphones. Throughout this process, the subject may be connected to a pulse oximeter device to capture ground truth PPG. They also may take demographic information that may affect the reading, including age, height, weight, biological sex, hand size, and skin tone. To ensure accuracy of the BP reading, BP may be measured via auscultation and a digital stethoscope may be used along with a webcam. The webcam video may be spliced with stethoscope audio. A data collection application may record fingertip videos. This application may record fingertip video with frame-by-frame metadata that includes the current camera settings for each frame. To align the PPG reading and a smartphone fingertip reading, a breadboard that has a button hooked up to an LED (e.g., a red LED) and to the pulse oximeter may be used. The button activates the LED and sends a signal to the PPG device to start recording. The smartphone is held up to the LED and the smartphone and PPG signals may be aligned based on that light. The data collection process may be mobile to enable capture of training data from a greater diversity of subjects by going to them on-site rather than them having to come to a specific location. Once data is collected, it may be uploaded into cloud storage. Processing the data may begin once it's uploaded, automatically syncing the PPG and smartphone fingertip reading (by looking for a spike in the red), clipping the smartphone and PPG signals to just the relevant portions with the fingertip on the camera (by looking for ratio of red to blue and green), fine-tuning the alignment of the PPG and smartphone signal, scoring the smartphone signals, and producing plots that can be analyzed. This data can be used to train the model(s). In addition to preparing data and producing plots, a database may also be produced of individual pulses that include signal quality score info and demographic data that we can use to debug a model quickly. The system may potentially use the demographic data as input into the BP model for greater accuracy as well, and for segmenting users to provide more accurate BP values.

In some embodiments, the system or services described herein may be implemented as microservices, processes, workflows or functions performed in response to the submission of a set of input data. The microservices, processes, workflows or functions may be performed by a server, data processing element, platform, or system. In some embodiments, the data analysis and other services may be provided by a service platform located “in the cloud.” In such embodiments, the platform may be accessible through APIs and SDKs. The functions, processes and capabilities described herein and with reference to one or more of the figures may be provided as microservices within the platform. The interfaces to the microservices may be defined by REST and GraphQL endpoints. An administrative console may allow users or an administrator to securely access the underlying request and response data, manage accounts and access, and in some cases, modify the processing workflow or configuration.

Note that although some embodiments described herein are directed to a multi-tenant or SaaS architecture that may be used for the delivery of business-related or other applications and services to multiple accounts/users, such an architecture may also be used to deliver other types of data processing services and provide access to other applications. For example, such an architecture may be used to provide one or more of the processes, functions, and operations described herein. Although in some embodiments, a platform or system of the type illustrated in the figures may be operated by a 3rd party provider to provide a specific set of services or applications, in other embodiments, the platform may be operated by a provider and a different entity may provide the applications or services for users through the platform.

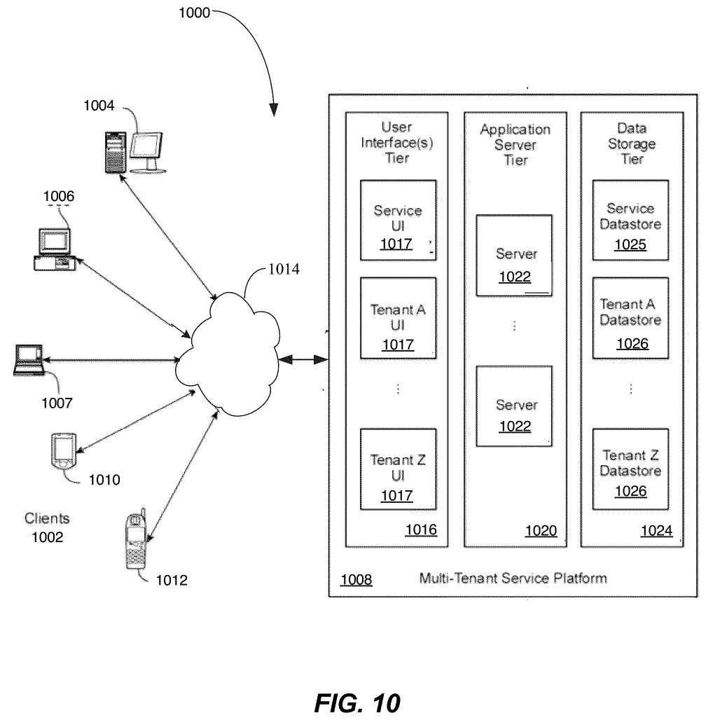

FIG. 9 illustrates an SaaS system 900 in which an embodiment may be implemented or through which an embodiment of the services described herein may be accessed. In accordance with the advantages of an application service provider (ASP) hosted business service system (such as a multi-tenant data processing platform), users of the services described herein may comprise individuals, businesses, stores, organizations, etc. A user may access the services using any suitable client, including but not limited to desktop computers, laptop computers, tablet computers, scanners, smartphones, etc. In general, any client device having access to the Internet may be used to provide data to the platform for processing and evaluation. A user interfaces with the service platform across the Internet 908 or another suitable communications network or combination of networks. Examples of suitable client devices include desktop computers 903, smartphones 904, tablet computers 905, or laptop computers 906.

System 900, which may be hosted by a third party, may include a set of data analysis and other services to assist in acquiring and processing video data, and generating a measure or measures of one or more health-related quantities, and a web interface server 914. The services may include one or more functions or operations for the processing of acquired video or other data to enable the generation of a measure of blood pressure or other health-related quantity. As examples, in some embodiments, the set of functions, operations or services made available through the platform or system 900 may include Account Management services 916, which may implement a process or service to authenticate a user wishing to submit an example of video data and from that determine a measure of a health-related quantity, or which may implement a process or service to generate a container or instantiation of the data processing and analysis services for that user. System 900 may also include Initial Data Acquisition and Processing services 917, which may implement a process or service to provide a client-side application to a user for installation on their device, may implement a process or service to receive a set of frames of video data from the user device; or may implement a process or service to process each frame to extract a red or pseudo-PPG channel (the PPG Signal Generator)

System 900 may also include Identify/Detect Video Collection Issues of Possible Concern services 918, which may implement a process or service to monitor and detect one or more situations or events that might indicate the video data acquired during that situation or event is not reliable or accurate, or may implement a process or service to execute logic to control the collection or processing of the video frames based on the presence or absence of such a situation or event. System 900 may also include Determine Health Risks, such as Blood Pressure Measure(s) and/or Other Health-Related Quantities services 919, which may implement a process or service to assess environmental factors and signal quality factors (in one embodiment using a trained signal quality assessment model), and if those are acceptable, to provide the generated (extracted) PPG signal to a trained model. Determine Health Risks Measure(s) and/or Other Health-Related Quantities services 919 may also implement a trained model that operates to receive the generated PPG signal extracted from the video frames and in response to generate a measure or measures of one or more health related quantities, such as blood pressure.

System 900 may also include Control Logic services 920, which may implement a process or service to implement other needed control logic to control the acquisition, processing, and use of the trained model(s), may implement a process or service to implement other logic for delivering and presenting the output of the trained blood pressure model to a user, including trend data and/or other forms of analysis of blood pressure measures, or may implement a process or service to implement logic to process data from other sensors and/or to process such data to generate other forms of signals for input to a trained model. System 900 may also include administrative services 922, which may implement a process or services to provide platform and services administration, such as, for example, enabling the provider of the services and/or the platform to administer and configure the processes and services provided to users.

The platform or system illustrated in FIG. 9 may be hosted on a distributed computing system made up of at least one, but likely multiple, “servers.” A server is a physical computer dedicated to providing data storage and an execution environment for one or more software applications or services intended to serve the needs of the users of other computers that are in data communication with the server, for instance via a public network such as the Internet. The server, and the services it provides, may be referred to as the “host” and the remote computers, and the software applications running on the remote computers being served may be referred to as “clients.” Depending on the computing service(s) that a server offers it could be referred to as a database server, data storage server, file server, mail server, print server, web server, etc. A web server is most often a combination of hardware and the software that helps deliver content, commonly by hosting a website, to client web browsers that access the web server via the Internet.

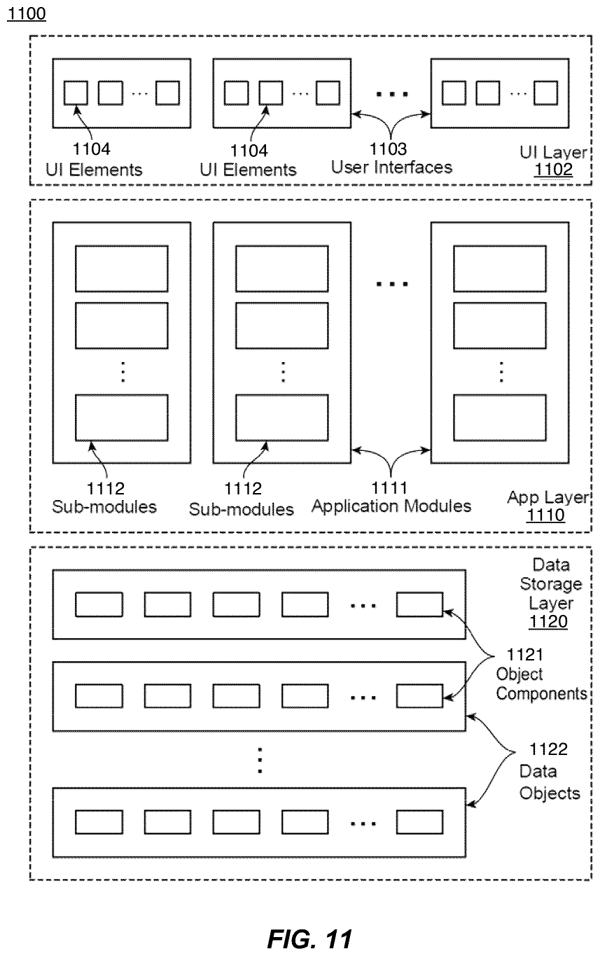

FIG. 10 illustrates elements or components of an example operating environment 1000 in which embodiments described herein may be implemented. As illustrated, a variety of clients 1002 incorporating and/or incorporated into a variety of computing devices may communicate with a multi-tenant service platform 1008 through one or more networks 1014. For example, a client may incorporate and/or be incorporated into a client application (e.g., software) implemented at least in part by one or more of the computing devices. Examples of suitable computing devices include personal computers, server computers 1004, desktop computers 1006, laptop computers 1007, notebook computers, tablet computers or personal digital assistants (PDAs) 1010, smart phones 1012, cell phones, and consumer electronic devices incorporating one or more computing device components, such as one or more electronic processors, microprocessors, central processing units (CPU), or controllers. Examples of suitable networks 1014 include networks utilizing wired and/or wireless communication technologies and networks operating in accordance with any suitable networking and/or communication protocol (e.g., the Internet).

The distributed computing service/platform (which may also be referred to as a multi-tenant data processing platform) 1008 may include multiple processing tiers, including a user interface tier 1016, an application server tier 1020, and a data storage tier 1024. The user interface tier 1016 may maintain multiple user interfaces 1017, including graphical user interfaces and/or web-based interfaces. The user interfaces may include a default user interface for the service to provide access to applications and data for a user or “tenant” of the service (depicted as “Service UI” in the figure), as well as one or more user interfaces that have been specialized/customized in accordance with user specific requirements (e.g., represented by “Tenant A UI”, . . . , “Tenant Z UI” in the figure, and which may be accessed via one or more APIs).

The default user interface may include user interface components enabling a tenant to administer the tenant's access to and use of the functions and capabilities provided by the service platform. This may include accessing tenant data, launching an instantiation of a specific application, causing the execution of specific data processing operations, etc. Each application server or processing tier 1022 illustrated in the figure may be implemented with a set of computers and/or components including computer servers and processors, and may perform various functions, methods, processes, or operations as determined by the execution of a software application or set of instructions. The data storage tier 1024 may include one or more data stores, which may include a Service Data store 1025 and one or more Tenant Data stores 1026. Data stores may be implemented with any suitable data storage technology, including structured query language (SQL) based relational database management systems (RDBMS).

Service Platform 1008 may be multi-tenant and may be operated by an entity to provide multiple tenants with a set of business-related or other data processing applications, data storage, and functionality. For example, the applications and functionality may include providing web-based access to the functionality used by a business to provide services to end-users, thereby allowing a user with a browser and an Internet or intranet connection to view, enter, process, or modify certain types of information. Such functions or applications are typically implemented by one or more modules of software code/instructions that are maintained on and executed by one or more servers 1022 that are part of the platform's Application Server Tier 1020. As noted with regards to FIG. 9, the platform system illustrated in FIG. 10 may be hosted on a distributed computing system made up of at least one, but typically multiple, “servers.”

As mentioned, rather than build and maintain such a platform or system themselves, a business may utilize systems provided by a third party. A third party may implement a business system/platform as described above in the context of a multi-tenant platform, where individual instantiations of a business' data processing workflow (such as the data analysis and evaluation services and processing described herein) are provided to users, with each business representing a tenant of the platform. One advantage to such multi-tenant platforms is the ability for each tenant to customize their instantiation of the data processing workflow to that tenant's specific business needs or operational methods. Each tenant may be a business or entity that uses the multi-tenant platform to provide business services and functionality to multiple users.

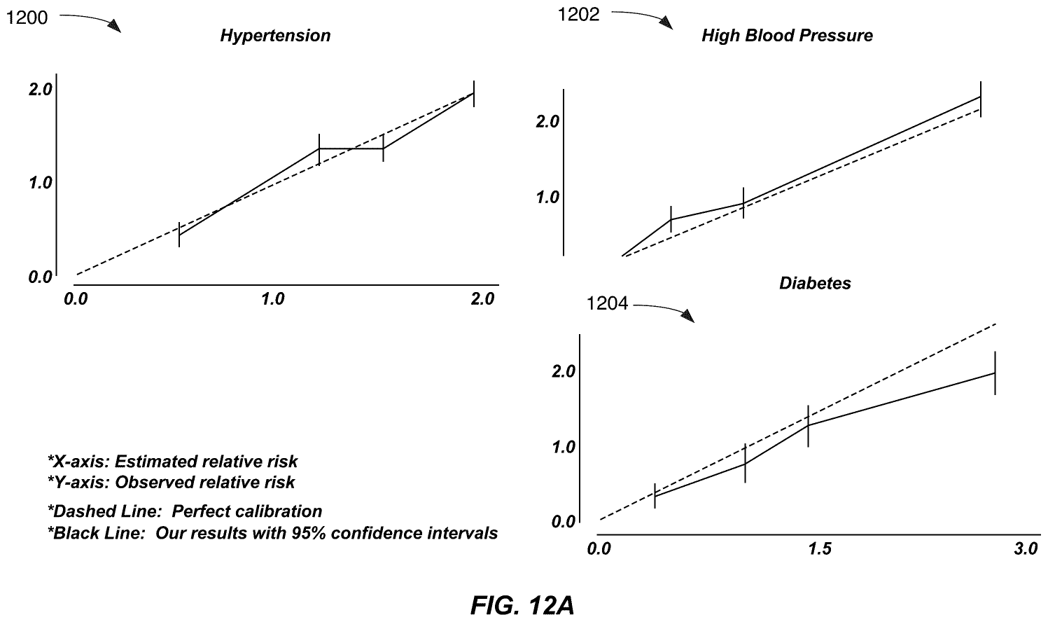

FIG. 11 illustrates additional details of the elements or components of the multitenant distributed computing service platform of FIG. 10, in which an embodiment may be implemented. The software architecture illustrated in FIG. 11 represents an example of an architecture which may be used to implement an embodiment. In general, an embodiment may be implemented using a set of software instructions that are designed to be executed by a suitably programmed processing element (such as a CPU, microprocessor, processor, controller, computing device, etc.). In a complex system such instructions are typically arranged into “modules” with each such module performing a specific task, process, function, or operation. The entire set of modules may be controlled or coordinated in their operation by an operating system (OS) or other form of organizational platform.

As noted, FIG. 11 is a diagram illustrating additional details of the elements or components 1100 of a multi-tenant distributed computing service platform, in which an embodiment may be implemented. The example architecture includes a user interface layer or tier 1102 having one or more user interfaces 1103. Examples of such user interfaces include graphical user interfaces and APIs. Each user interface may include one or more interface elements 1104. For example, users may interact with interface elements to access functionality and/or data provided by application and/or data storage layers of the example architecture. Examples of graphical user interface elements include buttons, menus, checkboxes, drop-down lists, scrollbars, sliders, spinners, text boxes, icons, labels, progress bars, status bars, toolbars, windows, hyperlinks, and dialog boxes. Application programming interfaces may be local or remote and may include interface elements such as parameterized procedure calls, programmatic objects, and messaging protocols.

The application layer 1110 may include one or more application modules 1111, each having one or more sub-modules 1112. Each application module 1111 or sub-module 1112 may correspond to a function, method, process, or operation that is implemented by the module or sub-module (e.g., a function or process related to providing business related data processing and services to a user of the platform). Such function, method, process, or operation may include those used to implement one or more aspects of the inventive system and methods, such as for one or more of the processes or functions described with reference to the figures.

The application modules and/or sub-modules may include any suitable computer-executable code or set of instructions (e.g., as would be executed by a suitably programmed processor, microprocessor, or CPU), such as computer-executable code corresponding to a programming language. For example, programming language source code may be compiled into computer-executable code. Alternatively, or in addition, the programming language may be an interpreted programming language such as a scripting language. Each application server (e.g., as represented by element 1022 of FIG. 10) may include each application module. Alternatively, different application servers may include different sets of application modules. Such sets may be disjoint or overlapping.

The data storage layer 1120 may include one or more data objects 1122 each having one or more data object components 1121, such as attributes and/or behaviors. For example, the data objects may correspond to tables of a relational database, and the data object components may correspond to columns or fields of such tables. Alternatively, or in addition, the data objects may correspond to data records having fields and associated services. Alternatively, or in addition, the data objects may correspond to persistent instances of programmatic data objects, such as structures and classes. Each data store in the data storage layer may include each data object. Alternatively, different data stores may include different sets of data objects. Such sets may be disjoint or overlapping.

Note that the example computing environments depicted in FIGS. 8-11 are not intended to be limiting examples. Further environments in which an embodiment may be implemented in whole or in part include devices (including mobile devices), software applications, systems, apparatuses, networks, SaaS platforms, IaaS (infrastructure-as-a-service) platforms, or other configurable components that may be used by multiple users for data entry, data processing, application execution, or data review.

Any of the software components, processes or functions described in this application may be implemented as software code to be executed by a processor using any suitable computer language such as Python, Java, JavaScript, C++ or Perl using conventional or object oriented techniques. The software code may be stored as a series of instructions, or commands in (or on) a non-transitory computer-readable medium, such as a random-access memory (RAM), a read only memory (ROM), a magnetic medium such as a hard-drive or a floppy disk, or an optical medium such as a CD-ROM. In this context, a non-transitory computer-readable medium is almost any medium suitable for the storage of data or an instruction set aside from a transitory waveform. Any such computer readable medium may reside on or within a single computational apparatus and may be present on or within different computational apparatuses within a system or network.

According to one example implementation, the term processing element or processor, as used herein, may be a central processing unit (CPU), or conceptualized as a CPU (such as a virtual machine). In this example implementation, the CPU or a device in which the CPU is incorporated may be coupled, connected, and/or in communication with one or more peripheral devices, such as display. In another example implementation, the processing element or processor may be incorporated into a mobile computing device, such as a smartphone or tablet computer.

The non-transitory computer-readable storage medium referred to herein may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, synchronous dynamic random access memory (SDRAM), or similar devices or other forms of memories based on similar technologies. Such computer-readable storage media allow the processing element or processor to access computer-executable process steps, application programs and the like, stored on removable and non-removable memory media, to off-load data from a device or to upload data to a device. As mentioned, with regards to the embodiments described herein, a non-transitory computer-readable medium may include almost any structure, technology, or method apart from a transitory waveform or similar medium.

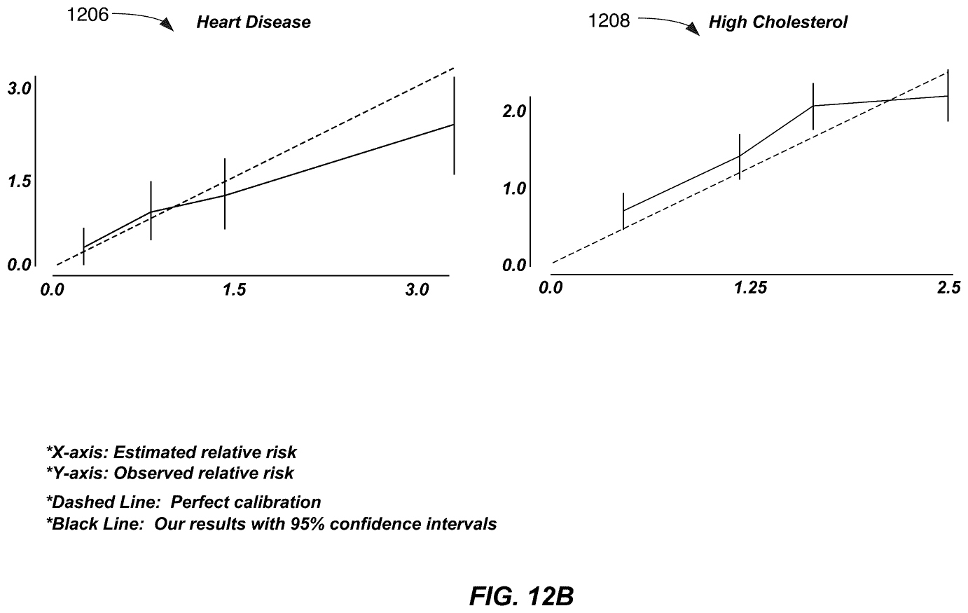

FIGS. 12A-16 relate specifically to using measured biosignals to assess a patient's health risks and/or predict health events, outcomes, and/or conditions. Some of these signal acquisitions can employ the quality signal acquisition techniques described above, such as acquiring a high quality PPG signal. These health assessment and prediction techniques provide next generation predictive and preventive healthcare by using accurate physiological information. The combination of acquired biosignals, such as a PPG signal acquired by a patient's smartphone, deep learning, and data science techniques provide valuable, physiological insight into a patient's risk of health conditions and events. This information allows data to drive healthcare decisions by prioritizing at-risk patients and intervening accordingly with preventive care. In nearly all these patients, they would not be identified as a health risk or the health event would occur for them in the conventional healthcare approach before intervention treatment and prevention could otherwise occur.

The closer a patient's biosignal signature looks to another patient that suffers from a health risk or condition or has experienced a health event, the higher the patient's risk of experiencing the health risk, condition, or event. FIGS. 12A and 12B show calibration curves for some health conditions-hypertension 1200, high blood pressure 1202, diabetes 1204, heart disease 1206, and high cholesterol 1208—in which the observed relative risk of the patient is compared to the estimated relative risk of the condition among the group of test patients by using the disclosed techniques. The dashed lines in each graph for a respective health condition represents a perfect calibration that predicts a health condition in test patients. The solid lines in each graph for the respective health condition represents the prediction of the health condition using the disclosed models. A comparison of the perfect calibration to the prediction by the model shows a high degree of accuracy in the model technique.

To create the graphs shown in FIGS. 12A and 12B, the patient's relative risk or “prevalence” of developing the health condition or suffering the health event is calculated, which is the predicted risk divided by the percentage of patients with that condition in the full reference population. The self-reported hypertension, high blood pressure, diabetes, high cholesterol, and heart disease diagnosis for all test patients was estimated using a nearest neighbors technique from a validation dataset of reference patients. A target patient's relative risk or prevalence was calculated, which is the percentage of patients with a health condition in each patient's neighborhood divided by the percentage of patients with that condition in the full reference population. For example, if 50% of patients in the full reference population have hypertension, but 75% of a patient's neighbors have hypertension, then the relative risk would be 0.75/0.5=1.5. The test patients were evenly sorted into four quartiles according to their estimated relative risk. For each of the quartiles, the observed relative risk is calculated, which is the percentage of patients in each quartile that actually have a health condition as compared to the full reference population. The graphs in FIGS. 12A and 12B shows that the more a patient's PPG signal (or alternatively other biosignal(s)) looks like the typical blood flow signal (or other alternative biosignal(s)) of reference patients that suffer from a certain medical condition, the higher is the risk for that patient to also suffer from that same condition.

The disclosed techniques rely on one or multiple measured biosignals that are used to create a physiological signature for the target patient that is an accurate representation of the patient's physiological state and produces a unique physiological signature for the patient represented by a target patient embedding vector. Multiple organ systems (renal, cardiovascular, respiratory, and nervous, for example) are involved in regulating blood flow. Therefore, changes in the functioning of these organ systems impacts the blood flow and manifests in certain biosignals measured on a target patient, such as the target patient's PPG signal. An increase or decrease in blood pressure (BP), blood glucose levels, vascular age, breathing rate, heart rate variability, atherosclerosis, and other health conditions cause detectable changes in a target patient's biosignal, such as the target patient's PPG signal. Sometimes, a single biosignal can detect such changes while other times multiple biosignals, such as a PPG signal and a recorded vocal signal of a target patient together detect such changes in the form of a feature or characteristic change from a reference group.

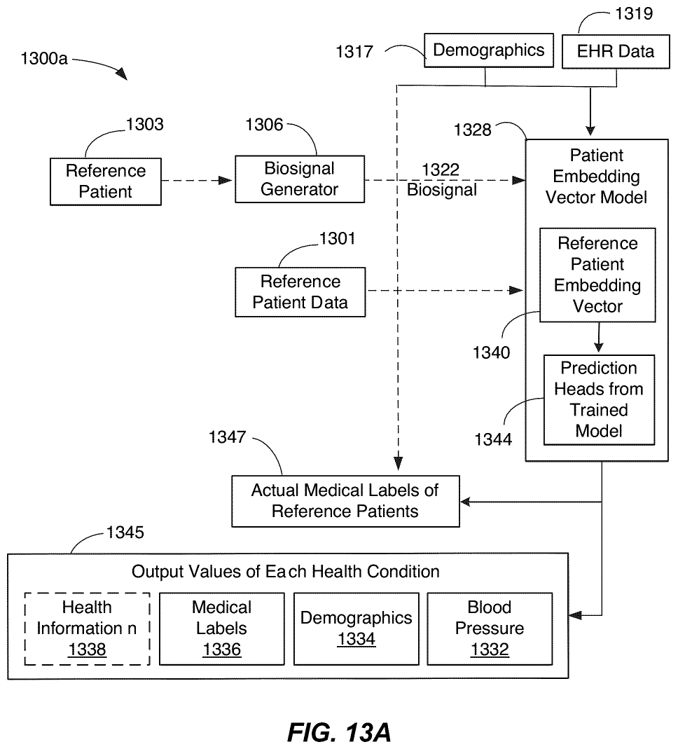

FIGS. 13A and 13B show systems and methods to train a patient embedding vector model, then use the model to create a strong embedding vector for a target patient to assess patient health risks and predict health events. Specifically, FIG. 13A is a block diagram showing a system 1300a and process flow with functional elements that train a patient embedding vector model to produce output values 1345 for reference patients for one or more patient health conditions, disease states, demographics, medical labels, or other health information. Actual medical labels or values of specific health conditions and confirmation that the reference patient suffers a health condition and associated values or their physiological state can also be calculated for each health condition/health event suffered by each reference patient. In some examples, the reference patient data is actively gathered from a reference patient 1303 while in other examples the data is received from another source that previously measured the reference patient data 1301. For the reference patient data that is actively gathered, the system has a biosignal generator 1306 that generates a biosignal 1322 of the reference patient 1303 that is then ingested into the patient embedding vector model 1328. Reference patient demographics 1317 and reference patient electronic health records data 1319 can also be ingested into the patient embedding vector model 1328 in some embodiments. Reference patient demographics 1317 relates to a patient's age, gender, weight, etc. The EHR data 1319 relates to any information in a patient's electronic health record or claims, which can help validate a health condition detected by one or more of the measured biosignal(s) in some examples. The system 1300a also can have reference patient data 1301 from other sources or stored by another device or server that is then ingested to the patient embedding vector model 1328.

For each reference patient, the system 1300a creates one or more reference patient output values 1345 for one or more health conditions and/or health events. The reference patient output values 1345 are numerical values that are used to train the patient embedding vector model 1328. During training, the patient embedding vector model 1328 generates a reference patient embedding vector 1340 based on the biosignal 1322 that includes any present health conditions, such as blood pressure 1332; certain demographics 1344 like age, gender, weight, etc.; medical labels 1336 such as diagnosed health conditions, data from EHRs or laboratory results; and any other health information n 1338 related to the reference patient. A biosignal, such as a PPG signal, includes information about a patient's blood pressure, demographics, and medical labels. For example, features and/or characteristics of a PPG signal of an older patient differ from the PPG signal of a younger patient. Similarly, a patient with hypertension has a PPG signal with different features and/or characteristics than a patient without hypertension. Multiple biosignals can be generated from the same or multiple sources in alternative embodiments (not shown). Any suitable number of reference patient biosignals can be generated. The system 1300a uses prediction heads 1344 of the patient embedding vector model 1328 to calculate output values 1345 for each reference patient with an identified health condition or health event.

For each reference patient, the patient embedding vector model 1328 outputs reference patient output values 1345, which are used to help predict whether a reference patient has a health condition or has experienced a health events. The output values 1345 help train the model to output a strong embedding vector of a target patient, as will be discussed further in reference to FIG. 13B. These output values 1345 are used to train the patient embedding vector model 1328 but are not used to identify health conditions or health events in a target patient. The actual medical labels 1347, such as a numerical value, identify whether a reference patient actually has a particular health condition or has experienced a particular health event. To train the patient embedding vector model 1328, the output values 1345 of each reference patient's health condition(s)/health event(s) are compared to the actual medical labels 1347 to compute the model loss. The actual medical labels 1347 are also used to later identify a prevalence of a health condition or health event in a target patient, as described in FIG. 13B.

The reference patient embedding vector 1340 uses the prediction head(s) 1344 of the trained patient embedding vector model 1328. Each prediction head 1344 correlates to a different health condition and/or health event and operates on the reference patient embedding vector 1340 for the reference patient to calculate the one or more output values 1345 related to the reference patient's physiological state. Each prediction head 1344 ingests the full reference patient embedding vector 1340 or a subset of the features. The prediction heads 1340 are each a series of one or more machine learning (ML) or deep learning layers, which could be as simple as logistic regression operating on the embedding vector or a more complex technique like multiple linear deep learning layers like multi-layer perceptron. Regardless of the ML technique used, the output from each prediction head can be a single output value 1345 related to the reference patient's health condition and/or health event, such as a patient's age, or multiple values, such as age, gender, weight, etc., as shown in FIG. 13A. It is these output values of each health condition 1345 for the reference patients that are used to train the patient embedding vector model 1328 by comparing the output values to the actual medical labels 1345 to create the model's training loss. The reference patient embedding vectors 1340 created during training of the patient embedding vector model 1328 help the system 1300b identify health conditions in the target patient embedding vectors. Actual medical labels 1347 can be identified through EHR data 1319 or reference patient data 1301 such as self-reported clinical diagnoses or biomarkers such as blood pressure measurements.

FIG. 13B shows a block diagram illustrating a system and process flow and functional elements that may be used in implementing various embodiments disclosed herein relating to assessing patient health risk and predicting health events using biosignals and deep learning models from a trained model, such as the trained patient embedding vector model 1328 of FIG. 13A. The disclosed system and process flow create an embedding vector representing a target patient's physiological state or physiological signature represented by a target patient embedding vector 1330 that is used to assess and/or predict a patient's health risk(s) and/or health event(s). The system 1300b shown in FIG. 13B uses training and validation data to optimize a deep learning model that takes one or more biosignals as input and estimates the patient's health information, such as demographics, blood pressure measurements, and self-reported medical labels. The deep learning model learns to condense the information in the biosignal(s) that is correlated to the patient's health information into a target patient embedding vector. This target patient embedding vector 1330 provides a concise numerical representation of a patient's physiological health and enables a flexible, standardized, and interpretable way to robustly estimate the health risk of a patient or predict health events for the patient. The target patient embedding vector 1330 is compared to the embedding vectors of the reference patients 1343 to identify various health conditions that the patient has, is likely to develop, or to identify health events that the patient may experience. The actual medical labels of reference patients 1347 help to calculate a prevalence of the identified health conditions in the target patient.

The system 1300b to assess a target patient's health risk and/or predict health events relies on a client application 1302 like the embodiment shown in FIG. 3. The systems 1300a, 1300b shown in FIGS. 13A and 13B, respectively, may be a DMM that includes sub-systems, elements, components, or modules that implement functionality for measuring blood pressure, among other biomarkers. Specifically in FIG. 13B, the system 1300b may include a client application 1302, a biosignal detector user interface (biosignal detector UI) 1304, and a biosignal generator 1307, as discussed above. Client application 1302 may be similar to or the same as the client application 302 described above. In the example of a PPG signal, the biosignal detector UI 1304 provides tools to a patient user to enable them to initiate the video/data collection used to determine their PPG signal from which various health condition/event data can be learned, similar to the Biomarker Detector UI 304 of FIG. 3. The biosignal detector UI 1304 controls or passes data or instructions to the biosignal generator 1307 to measure the biosignal in a similar way that the Biomarker Detector UI 304 controls the PPG Signal Generator 306. The biosignal detector UI 1304 and the biosignal generator 1307 may be coupled together in the same component 1308.

The system 1300b can have multiple biosignals 1324, 1326 controlled by the client application 1302. Multiple biosignals can include vocal signals and/or physiological signals or data from the target patient. Each additional biosignal 1324, 1326 can be produced by a biosignal component 1314, 1320 that includes a biosignal detector UI 1310, 1316 and a biosignal generator 1312, 1318 that respectively produce the multiple biosignals 1324, 1326. Any suitable number of biosignals can be produced to be ingested by the patient embedding vector model 1328. These biosignals 1322, 1324, 1326 are actively generated for target patients to create a strong patient embedding vector for the target patient 1330. The patient embedding vector model 1328 can additionally ingest patient demographics 1321 and EHR data 1323 from other sources than the biosignal components 1308, 1314, 1320.

The biosignal components 1308, 1314, 1320 can have different sources, such as different sensors measuring data on the target patient. Alternatively or additionally, one or more components can be housed together. For example, component 1308 could produce a first biosignal 1322, component 1314 could produce a second biosignal 1324, and component 1320 could produce a third biosignal 1326. Any suitable number of biosignal components can be included in the system 1300. The system 1300 transmits each of the one or more biosignals 1322, 1324, 1326 to the patient embedding vector model 1328 that generates a target patient embedding vector 1330 for a new, target patient. Further, the target patient embedding vector 1330 for the target patient can be later used to further train the patient embedding vector training model for future target patients in some example systems. The system 1300 can continuously train the patient embedding vector model 1328 on each target patient embedding vector that is created so the model continues to evolve and learn with each target patient assessment in an iterative manner, i.e., it continuously improves its ability to create strong target patient embedding vectors with each new target patient it assesses.

The patient embedding vector model 1328 ingests multiple biosignals 1322, 1324, 1326 in the system 1300 shown in FIG. 13B from one or more sources including but not limited to the client application 1302. The patient embedding vector model 1328 also can optionally ingest data about a patient's demographics 1321 and/or from a patient's electronic health record (EHR) 1323. These and other additional data sources for patient information can come through the client application 1302, but they are more likely to be ingested from another source, such as a server that stores the EHR for the patient and/or a demographics module separate from the client application.

The biosignals 1322, 1324, 1326 can be a PPG signal, a vocal signal, and any other biosignal measuring a patient's physiological state. From any one, multiple, or all of the ingested biosignals 1322, 1324, 1326, the patient embedding model 1328 identifies features or characteristics of the respective biosignal that correlate to a specific health condition, such as blood pressure, patient demographics, medical labels, or any other health information about the target patient. The patient embedding vector model 1328 first creates a target patient embedding vector 1330, then calculates a prevalence for each of these health conditions from the actual medical labels of the reference patients 1347. The system 1300b predicts risk of a particular health condition or health event by comparing the target patient embedding vector 1330 to the reference patient embedding vectors 1343. Once health condition(s) or health event(s) are identified in the target patient embedding vector 1330 through its comparison to the reference patient embedding vectors 1343, the system 1300b calculates the prevalence of the identified health condition using the actual medical labels from the reference patients.

Differences or changes in features or characteristics of any one, a combination, or all of the biosignal indicate a physiological state of the patient and, in some cases, multiple physiological states of the patient. From these changes measured in the biosignal, any suitable volume of health information can be generated by the patient embedding vector model 1328 if the correlating health condition is present in the target patient.

Each target patient assessed for health risks and/or for whom health events may be predicted can be added as a new training dataset for subsequent target patients so the model 1328 iteratively improves its ability to identify health conditions and/or health events and calculate their prevalence in future target patients. The patient embedding vector model 1328b must first be trained, as discussed above in reference to FIG. 13A, by using ingested biosignals from reference patients with known or suspected health conditions to predict various health conditions, such as blood pressure 1332, patient demographics 1334, and medical labels 1336 (e.g., self-reported or provider reported diagnoses of medical conditions). In some examples, medical providers can confirm health conditions for reference patients (and new patients being assessed) by inputting such diagnosed health data for a patient or by linking an electronic health record as input to the model.

After the trained patient embedding vector model 1328 produces the target patient embedding vector 1330 for the target patient, the target patient embedding vector 1330 and the reference patient embedding vectors 1343 are ingested to a nearest neighbor module 1342 that determines the nearest neighbors in the reference patient population. To identify the nearest neighbors, the nearest neighbor module 1342 compares the target patient embedding vector to the reference patient embedding vectors to create a neighborhood of nearest reference patients with similar embedding vectors to the target patient. Similar embedding vectors are numerical representations that correlate to a similar physiological state between the target patient and the reference patient(s). This neighborhood is then used to calculate the prevalence of each present health condition by using the actual medical labels 1347, as discussed further below. For some values, it may calculate an alternative aggregated metric, such as the average, for calculating demographics like age or weight or biomarkers like blood pressure. When the nearest neighbor module 1342 identifies the nearest neighbors to the target patient, it then outputs this data to the predicted health risk/event model 1346 that calculates the prevalence or aggregated metric of each identified health condition or health event by using the actual medical labels 1347 of the reference patients that are within the target patient's neighborhood. The health risk/event prediction model 1346 outputs an identification of the particular health conditions and/or health events that a target patient has or is likely to develop or experience along with an estimate of likelihood that such condition or event will occur. For some values such as biomarkers or demographics, it may output a value, such as age or systolic blood pressure.

The nearest neighbors to the target patient embedding vector 1330 have similar embedding vectors to the target patient. The similarity for a target patient embedding vector 1330 for a patient likely to suffer from high blood pressure, for example, will include nearest neighbors that also suffer from high blood pressure. Likewise, a young target patient or a target patient with diagnosed diabetes has a similarity in their target patient embedding vector 1330 to the embedding vectors of other young or diabetic patients, respectively, when the target patient embedding vector 1330 is used to identify a neighborhood of reference patients each having a respective reference patient embedding vector.

In some examples, when the patient embedding vector model 1328 generates the target patient embedding vector 1330 for a patient with multiple health conditions, the model 1328 can first generate individual embedding vectors for each of the health conditions detectable in the target patient biosignal(s). Those individual, condition-specific embedding vectors can then be combined using either a concatenation or aggregation technique, as desired. This technique of combining individual condition-specific data (whether or not in the form of an embedding vector) produces a target patient embedding vector 1330 that can be ingested into the nearest neighbor module 1342 with the reference patient embedding vectors and output values 1343 to be compared against reference patient embedding vectors for patients with one or more of the same health conditions as the target patient, for example. This allows the health risk/event prediction model 1346 to identify as many health conditions as the target patient has that are also present in the reference patient population and to predict a prevalence in the form of a numerical value for how likely the patient is to have or develop the health condition or experience the health event or a value for demographics and biomarkers like age and systolic blood pressure. The health risk/event prediction model 1346 is trained on the health conditions/events of the reference population. As target patients are assessed, they later can become part of the reference patient population for subsequent target patients, in some examples, as part of an iterative training process for the patient embedding vector model 1328.

For example, the system 1300b uses a nearest neighbor approach, such as the nearest neighbor module 1342 shown in FIG. 13B, to identify “n” reference patient embedding vectors 1344 that meet or exceed a certain criteria for comparing or matching the embedding vectors. One such criteria is selecting the nearest “n” reference patient embedding vectors 1344, regardless of distance. Similarity for indicating a health condition can vary based on specific needs, such as degree of certainty, physiological risk of the patient (e.g., medically fragile patients may have a lower threshold to identify possible health risks or events), or the like. Likewise, the threshold or criteria for “similar” embedding vectors may differ based on the health condition itself or on the volume of reference patients with a similar health condition.

Several types of nearest neighbor analyses can be used by the nearest neighbor module 1342 to determine a nearest neighbor for the neighborhood of the target patient. For example, the system 1300b can use a defined distance metric, such as a dot product, cosine similarity, etc. The system 1300b can also use a metric distance learning that learns a matrix to insert between the dot product to down-weight some of the individual dimensions of the target patient embedding vector 1330. To predict diabetes, for example, dimension 1 may be less important so it would have a lower weight to the overall distance between the dot products. In another example, the system 1330b could use a linear probe that uses linear regression like Ridge or Lasso to determine a distance between the target patient embedding vector 1330 and the reference patient embedding vector 1340. The system 1330b can also use a non-linear distance function like a multi-layer perceptron (MLP) or convolutional model in other examples.

To estimate the risk that the target patient suffers from a certain health condition, the system 1300b calculates such a prevalence of that condition within the target patient's “neighborhood.” The patient's neighborhood is the identified group of nearest neighbors from the reference patient population plotted by comparison of the target patient's embedding vector with the embedding vectors of the reference patients. The distance of the target embedding vector to a specific neighbor reference patient embedding vector is a quantifiable measurement of the similarities or differences between the respective embedding vectors, as discussed above. The greater number of differences between the respective embedding vectors, the greater the plotted distance between them on a graph or plot map of embedding vectors. Reference patient embedding vectors that are more similar or “closer” in distance to the target patient embedding vector have a higher likelihood of identifying a health risk or health event the target patient has or is likely to experience. The prevalence of nearest neighbors is the number of neighbors that have experienced a given health event divided by the total number of neighbors. The prevalence may also be distance-weighted, where the contribution of each neighbor to the prevalence calculation is based on their distance from the target embedding vector.

Those health conditions or health events with a higher prevalence will have a higher likelihood of the health condition or health event occurring in the target patient. This process of identifying the nearest neighbors to a target patient embedding vector, then calculating the prevalence, provides an accurate risk-estimate for whether a patient suffers from or will develop a particular health condition or is likely to experience a particular health event. The mapping is the same across all identified health conditions and health events although the specific assessment for which reference patients are within a target patient's nearest neighbor dataset can differ between health conditions and/or health events or can differ if considering combinations of various health conditions and/or health events.

The predicted health risk/event model 1346 takes the actual medical labels 1347 for each of the nearest neighbors then makes a prediction for the corresponding health condition/event of the target patient. For example, the predicted health risk/event model 1346 determines the age of the target patient by calculating the average of the actual age of the nearest neighbors from the reference patient actual ages 1347. Likewise with diabetes status, to determine the prevalence of diabetes in the target patient, the predicted health risk/event model 1346 sums the number of nearest neighbors having a diabetes diagnosis 1347 and divides that number by the total number of nearest neighbors to output the prevalence that the target patient has diabetes. The predicted health risk/event model 1346 may transform the prevalence or aggregated metric, based on the application, to a different scale to produce the risk score or final value. For example, it may be scaled between 0-1 or normalized to have unit variance.

The predicted biomarker/health risk/event prediction model 1346 outputs one or more predicted biomarkers/health risk(s)/event(s) for the patient based on the health conditions/health events in the nearest neighbor analysis. This output from the biomarker/health risk/event prediction model 1346 is an assessment of the patient's biomarkers such as blood pressure, health risks, and/or a prediction of a health event the patient is likely to experience based on the health conditions and health events in the identified nearest neighbors. Such predicted health risks can be a likelihood of the target patient developing a particular health condition or disease or experiencing a certain health event, such as a heart attack, cardiac arrhythmia, stroke, or the like. These predicted health risk(s)/health event(s) can be transmitted to the client application 1302 for the patient and/or to a medical provider application 1348 for a medical provider. The disclosed techniques and system provide scalable, real-time health information about a patient to medical providers to help identify health risks and intervene before the patient experiences harmful and expensive medical events or conditions.

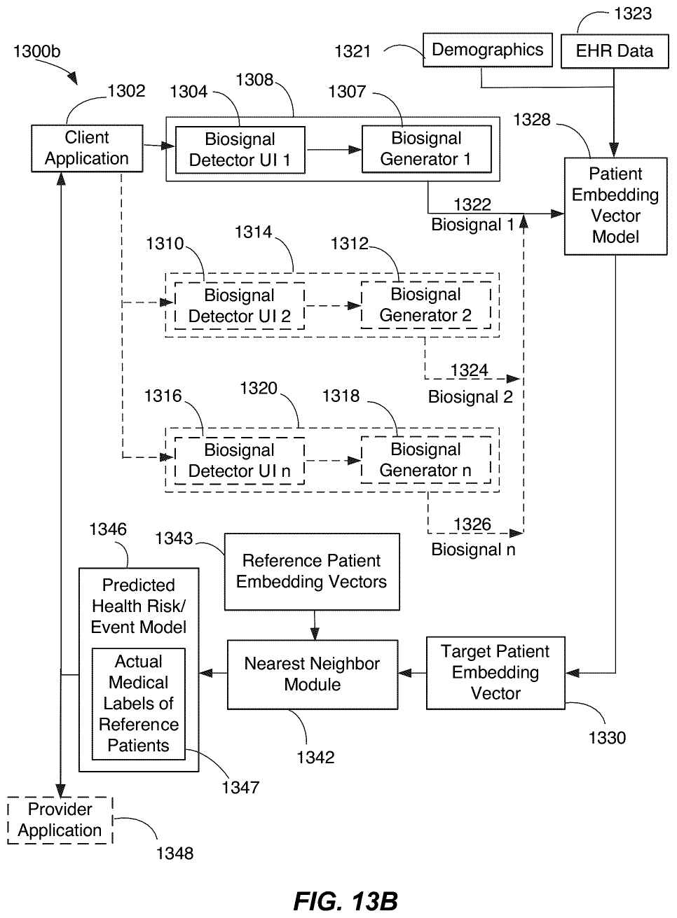

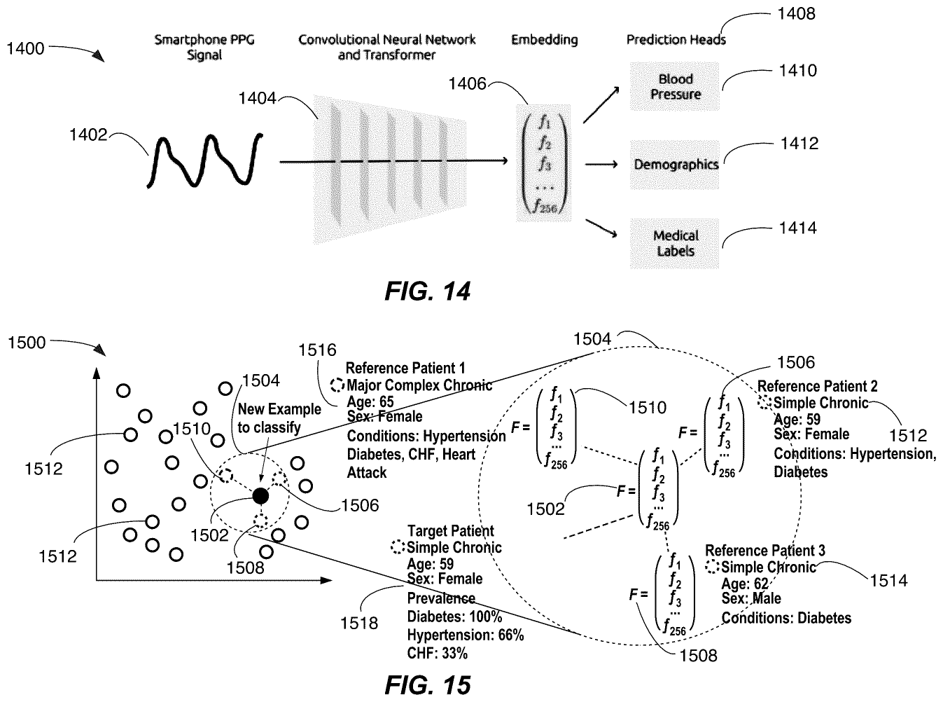

For example, a system, such as the system 1300 shown in FIGS. 13A and 13B, can measure a patient's PPG signal using a camera on the patient's smartphone, as discussed above. Other methods of acquiring a patient's PPG signal can also be used, such as a conventional pulse oximeter, for example. Turning now to FIGS. 14 and 15, FIG. 14 shows a deep learning model being trained 1400. After the model is trained 1400, the trained deep learning model 1400 can then ingest a target patient's PPG signal (or other biosignal in another example) to produce an embedding vector for the target patient. In FIG. 14, the biosignal is a patient PPG signal measured by the patient's smartphone 1402. To train the deep learning model 1400, the PPG signals are ingested 1402 into a convolutional neural network (CNN) and transformer 1404. The CNN and transformer 1404 are effective at extracting features and patterns from complex temporal signals. The CNN is particularly effective at finding patterns in 1-dimensional signals and transformers excel at mapping temporal signals. Combined, they can find features like the curvature of signals or presence of a dicrotic notch and their relation to similar features in other portions of the periodic signal. The CNN and transformer 1404 create an embedding vector 1406 from the ingested PPG signal 1402 for the target patient.

Depending on the features and/or characteristics found in the reference patient's PPG signal in this example, one or more prediction heads 1408 are identified and created. The prediction heads 1408 each include a series of one or more machine learning (or deep learning layers) that help identify a specific health condition from the embedding vector, such as high blood pressure 1410, various patient demographics 1412, and actual medical labels 1414 (e.g., diagnosed or self-reported medical conditions). For example, the PPG signal could indicate any one or more of high blood pressure, age, gender, weight, validated diabetes or other cardiovascular, respiratory, nephrology, endocrine, or nervous system conditions, etc. The example shown in FIG. 14, shows prediction heads 1408 of high blood pressure 1410, demographics 1412, and various medical labels 1414. These prediction heads 1408 are used to train the patient embedding vector model on multiple reference patients with similar sets of health conditions and are then used to create strong embedding vectors of target patients when used for health assessment and prediction to help in preventive care.

FIG. 15 shows a nearest neighbor assessment of a target patient 1500 based on a target patient embedding vector 1502 created by a patient embedding vector model, such as the trained deep learning model 1400 shown in FIG. 14. The nearest neighbor assessment 1500 identifies a neighborhood 1504 of the target patient using any of the disclosed criteria techniques to identify reference patients, based on their respective reference patient embedding vectors 1506, 1508, 1510. Other reference patients 1512 that are outside the neighborhood 1504 are removed from further assessment of the target patient. In the example shown in FIG. 15, three reference patients have embedding vectors that meet a criteria (e.g., are within a certain distance on a plot map) to be considered a neighbor of the target patient based on a comparison of the target patient embedding vector 1502 to the respective reference patient embedding vectors 1506, 1508, 1510.

The reference patients 1506, 1508, 1510 can be categorized into various patient groups based on their respective health states 1512, 1514, 1516. For example, reference patient 1 1510 is categorized as a major complex chronic patient based on their physiological signature 1516 based on their age, gender, and/or their number and type of health conditions. Reference patient 2 and reference patient 3 are both categorized as simple chronic patients based on their health states 1512, 1514 that include age, gender, and/or their number and type of health conditions. Within the reference patient embedding vectors 1506, 1508, 1510 within the neighborhood 1504, the reference patient embedding vector of reference patient 2 1506 is most similar to the target patient embedding vector 1502, which may be assigned a weighting higher than the reference patient embedding vector of reference patient 1 1510. The target patient health state 1518 of the target patient has a prevalence percentage likelihood of having or developing three health conditions-diabetes, hypertension, and congestive heart failure (CHF)-based on the target patient embedding vector 1502 similarities with the reference patient embedding vectors 1506, 1508, 1510. Notably, each of the health conditions listed in the target patient's health state 1518 are included in the respective health states 1512, 1514, 1516 of reference patient 1, reference patient 2, and reference patient 3. The likelihood that the target patient has or will develop diabetes is 100% with the reference patients in the neighborhood each having diabetes while the likelihood of the target patient developing hypertension being 66% with two of the three reference patients having hypertension and the likelihood of the target patient developing CHF being 33% with one of the three reference patients having CHF.

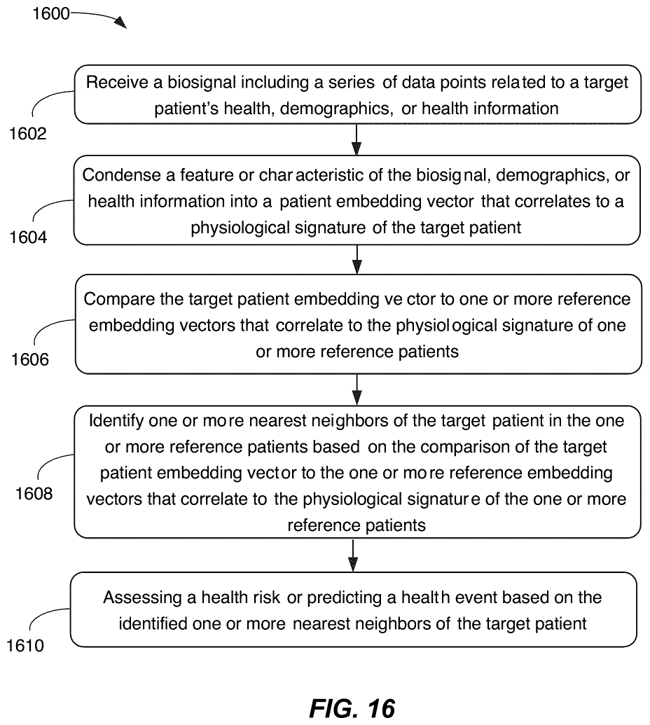

FIG. 16 shows a method of assessing health risk and predicting health events of a target patient using measured biosignals. The method receives a biosignal or multiple biosignals that include a series of data points related to a patient's health, demographics, or health information 1602, such as the biosignals and related health conditions discussed herein. A feature or characteristic of the biosignal, demographics, or health information are condensed into a patient embedding vector that correlates to a physiological signature of the target patient 1604. The target patient embedding vector is then compared to one or more reference embedding vectors that correlate to a respective physiological signature of one or more reference patients 1606. One or more nearest neighbors of the target are identified in the one or more reference patients based on the comparison of the target patient embedding vector to the one or more reference embedding vectors that correlate to the physiological signature of the one or more reference patients 1608. A health risk can be assessed and/or a health event can be predicted for the target patient based on the identified one or more nearest neighbors of the target patient 1610.

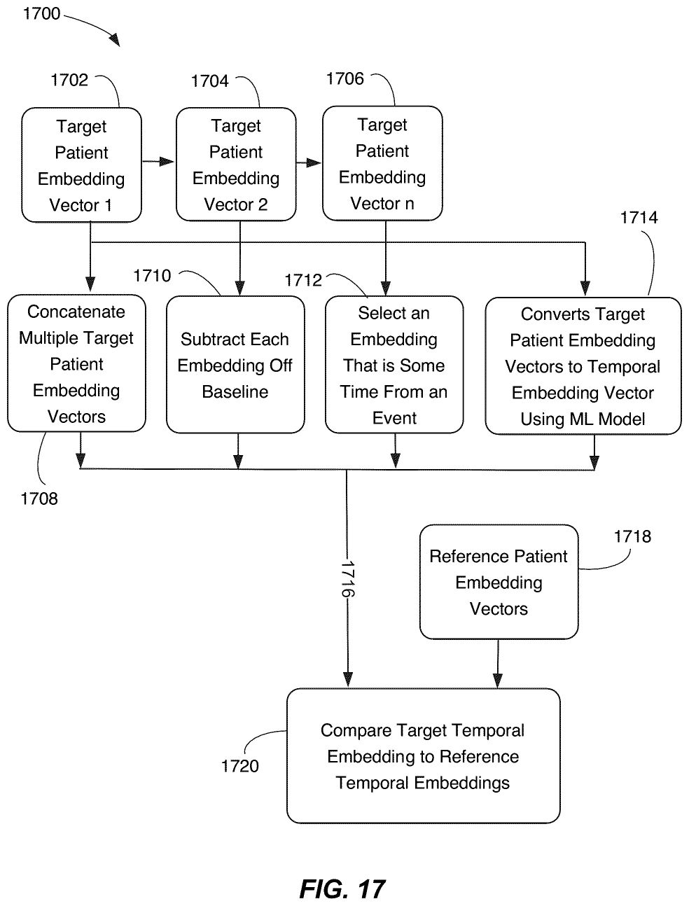

Turning now to FIG. 17, a flow diagram of a system 1700 is shown that finds nearest neighbors of the target patient that have similar temporal patterns. Temporal patterns are trends in a health condition or physiological state over time. For example, a temporal pattern post-operative of a heart surgery could identify post-operative atrial fibrillation or infection before it occurs, which are common health conditions to develop after certain heart surgeries like an open heart bypass surgery. Another example is the deterioration of recently discharged patients after a hospital visit, such as decompensation for a heart failure patient or the development of sepsis for a post-operative patient. The progression of target patient medical labels or monitoring vital signs like blood pressure over time that indicate certain health conditions are developing or improving can also be examples of temporal signals. This kind of temporal analysis gives the system 1700 a way to predict health events before they occur because other patients with similar temporal patterns from the reference population had similar temporal patterns before such an event.

Instead of analyzing just one event, multiple target patient embedding vectors are analyzed. These target patient embedding vectors correlate to events of the target patient that occur at discrete points in time over a time period. For example, target patient embedding vector 1 correlates to the day after the target patient was discharged from the hospital for a health condition. Target patient embedding vector 2 correlates to two days after the target patient was discharged from the hospital for the health condition. Target patient embedding vectors 1 and 2, respectively, are compared to embedding vectors of reference patients one and two days after they were discharged from the hospital for the same health condition. If the reference patient(s) experienced a health event on day 5 after being discharged from the hospital, and their embedding vectors on the day after and two days after hospital discharge are similar to those of the target patient on the same days after hospital discharge, then the target person can receive intervention treatment before the health event occurs on day 5 after the hospital discharge.

In some examples, the target patient has three embedding vectors 1702, 1704, 1706 taken at different times. One way to identify similar temporal patterns between a target patient and reference patients is to concatenate the target patient embedding vectors together 1708 to make one long target patient embedding vector (e.g., target patient embedding vector 1 is concatenated with target patient embedding vector 2). Normal distance metrics, as discussed above for similar or un-concatenated vectors, are used to compare target patient embedding vectors (concatenated or not) with reference patient embedding vectors. Also, alternatively or additionally, to identify a temporal pattern for each target patient embedding vector, each target patient embedding vector can be subtracted off a baseline 1710, such as a fixed day (i.e., day 1 after hospital discharge); an average (i.e., average values of last 7 days); or updated average (i.e., exponential moving average over the last 7 days).

Another way to identify similarities between target patient embedding vectors and reference patient embedding vectors is to select an embedding that is some time, such as a defined time period, from an event. For example, the system 1700 can analyze an embedding vector for a target patient that is 3 days out of surgery by comparing it to reference patient embedding vectors on day 3 after the same surgery for reference patients that experienced a particular health event. Often such adverse health events require readmission to the hospital or serious decline is a patient's physiological state requiring urgent or emergency care. Being able to predict these events and intervene with proper treatment before they happen save lives, improve quality of life, lower healthcare costs, and reduce the impact of adverse health events on families, communities, and health care systems.

Yet another way of identifying temporal similarities between target patient embedding vectors and reference patient embedding vectors is to convert a target patient embedding vector to a temporal embedding vector using an ML model 1714. The ML model analyzes the target patient embedding vectors over a certain defined time period, such as the previous 7 days, for example. Example ML models that can analyze the target patient embedding vectors in this way include linear models, state-space models, CCNs, RNNs, and transformers. Missing data (i.e., if there is no biosignal for one of the 7 days) may be imputed by using the surrounding data, such as an average between the embeddings from the day before and after, or using data from other biosignals. The output from such an ML model is a temporal embedding vector for the target patient that is compared to a temporal embedding vector for the reference patients. Any one or more of the above described ways to identifies similar temporal patterns between the target patient embedding vector and the reference patient embedding vectors can be used. The output of the similarity analysis 1716 is used in combination with reference patient embedding vectors 1718 to compare the target patient temporal embedding vector or temporal pattern to the reference patient temporal embedding or temporal patterns 1720.

Certain implementations of the disclosed technology are described herein with reference to block diagrams of systems, and/or to flowcharts or flow diagrams of functions, operations, processes, or methods. It will be understood that one or more blocks of the block diagrams, or one or more stages or steps of the flowcharts or flow diagrams, and combinations of blocks in the block diagrams and stages or steps of the flowcharts or flow diagrams, respectively, may be implemented by computer-executable program instructions. Note that in some embodiments, one or more of the blocks, or stages or steps may not necessarily need to be performed in the order presented or may not necessarily need to be performed at all.

These computer-executable program instructions may be loaded onto a general purpose computer, a special purpose computer, a processor, or other programmable data processing apparatus to produce a specific example of a machine, such that the instructions that are executed by the computer, processor, or other programmable data processing apparatus create means for implementing one or more of the functions, operations, processes, or methods described herein. These computer program instructions may also be stored in a computer readable memory that may direct a computer or other programmable data processing apparatus to function in a specific manner, such that the instructions stored in the computer-readable memory produce an article of manufacture including instruction means that implement one or more of the functions, operations, processes, or methods described herein.

Other objects and advantages of the systems and methods described will be apparent to one of ordinary skill in the art upon review of the detailed description and the included figures. Throughout the drawings, identical reference characters and descriptions indicate similar, but not necessarily identical, elements. While the exemplary embodiments described herein are susceptible to various modifications and alternative forms, specific embodiments have been illustrated by way of example in the drawings and will be described in detail herein. However, the exemplary embodiments described herein are not intended to be limited to the particular forms disclosed. Rather, the present disclosure covers all modifications, equivalents, and alternatives falling within the scope of the appended claims.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein may be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the embodiments unless otherwise claimed. No language in the specification should be construed as indicating any non-claimed element as essential to each embodiment.

As used herein (e.g., the claims, figures, and specification), the term “or” is used inclusively to refer items in the alternative and in combination.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific embodiments or examples are presented by way of examples for purposes of illustration and description. They are not intended to be exhaustive of or to limit this disclosure to the precise forms described. Many modifications and variations are possible in view of the above teachings. The embodiments or examples are illustrated and described in order to best explain the principles of this disclosure and practical applications, to thereby enable others skilled in the art to best utilize this disclosure and various embodiments or examples with various modifications as are suited to the particular use contemplated. It is intended that the scope of this disclosure be defined by the following claims and their equivalents.

1. A method of assessing a patient health risk or predicting a patient health event, comprising:
receiving a biosignal including a series of data points related to a target patient's health, demographics, or health information;
condensing a feature or characteristic of the biosignal related to the target patient's health, demographics, or health information into a target patient embedding vector that correlates to a physiological signature of the target patient;
comparing the target patient embedding vector to one or more reference embedding vectors that correlate to one or more reference patients;
identifying one or more nearest neighbors of the target patient in the reference patients based on the comparison of the target patient embedding vector to the one or more reference embedding vectors that correlate to the one or more reference patients; and
assessing the patient health risk or predicting the patient health event based on the identified one or more nearest neighbors of the target patient.

2. The method of claim 1, wherein the biosignal is a photoplethysmography (PPG) signal or a pseudo PPG signal.

3. The method of claim 1, wherein the biosignal is a video signal or a vocal signal.

4. The method of claim 1, wherein the biosignal includes one or more of a photoplethysmography (PPG) signal, a video signal, and a vocal signal.

5. The method of claim 1, wherein the series of data points is related to two or more of a patient's health, demographics, and health information.

6. The method of claim 5, wherein the series of data points related to the two or more of the patient's health, demographics, and health information is received from different sources.

7. The method of claim 1, wherein the series of data points is related to a patient's health, demographics, and health information.

8. The method of claim 7, wherein the series of data points related to the patient's health is condensed to a patient health embedding vector, the series of data points related to the patient's demographics is condensed to a patient demographics embedding vector, and the series of data points related to a patient's health information is condensed to a patient health information embedding vector.

9. The method of claim 8, further comprising combining the patient health embedding vector, the patient demographics embedding vector, and the patient health information embedding vector into the patient embedding vector using either concatenation or aggregation techniques.

10. The method of claim 9, wherein the combined patient health embedding vector, the patient demographics embedding vector, and the patient health information embedding vector are compared to multiple reference embedding vectors.

11. The method of claim 9, further comprising ingesting the combined patient health embedding vector, the patient demographics embedding vector, and the patient health information embedding vector into an embedding vector training model for reference patients.

12. The method of claim 11, further comprising training the embedding vector training model for reference patients with the combined patient health embedding vector, the patient demographics embedding vector, and the patient health information embedding vector.

13. The method of claim 11, wherein the embedding vector training model for reference patients also compares the target patient embedding vector to the one or more reference embedding vectors that correlate to the physiological parameter of the one or more reference patients.

14. The method of claim 13, further comprising:
for each of the reference patient's health conditions, demographics, and health information, producing a respective actual medical label;
comparing the target patient embedding vector to the one or more reference patient embedding vectors to identify at least one nearest neighbor of the target patient from the one or more reference patients;
calculate a prevalence or aggregated metric such as average of the target patient having the health conditions, demographics, and health information based on the actual medical labels of each reference patient identified as a nearest neighbor of the target patient.

15. The method of claim 1, wherein assessing the patient health risk or predicting the patient health event is further based on an actual medical label of one or more of the reference patients.

16. The method of claim 15, wherein the series of one of more machine learning layers includes one or more of a logistic regression operating on the patient embedding vector or multiple linear deep learning layers.

17. The method of claim 1, further comprising identifying multiple nearest neighbors of the patient from the reference patients.

18. The method of claim 17, further comprising calculating the value of a health quantity, including as biomarker or demographic, health risk, or health event, by using an aggregated value such as mean, prevalence, or distance-weighted prevalence within the identified multiple nearest neighbors of the patient.

19. The method of claim 1, further comprising:
condensing the feature or characteristic of the biosignal related to the patient's health, demographics, or health information into multiple patient embedding vectors that correlate to multiple respective physiological parameters of the patient;
comparing the multiple patient embedding vectors to multiple reference embedding vectors that correlate to multiple reference patients;
identifying multiple nearest neighbors of the patient in the multiple reference patients based on the comparison of the patient embedding vector to the multiple reference embedding vectors; and
assessing the patient health risk or predicting the patient health event based on the identified multiple nearest neighbors of the patient.

20. The method of claim 19, further comprising assessing multiple patient health risks, predicting multiple patient health events, or estimating values like demographics or biomarkers based on the identified multiple nearest neighbors of the patient.

21. The method of claim 1, wherein the one or more nearest neighbors of the target patient are calculated using one or more of a dot product, cosine similarity, metric distance learning, linear probe, non-linear probe distance function.

22. The method of claim 1, wherein the target patient embedding vector is a first target patient embedding vector at a first time, and further comprising:
receiving a biosignal including a series of data points related to the target patient's health, demographics, or health information at a second time;
condensing the feature or characteristic of the biosignal related to the target patient's health, demographics, or health information into a second target patient embedding vector that correlates to a physiological signature of the target patient at the second time;
determining a temporal pattern or temporal embedding vector for the target patient based on the first embedding vector and the second embedding vector; and
identifying nearest neighbors of the target patient based on the temporal pattern or the temporal embedding vector.

23. The method of claim 22, wherein the temporal pattern or temporal embedding vector further includes:
concatenating the first target patient embedding vector with the second target patient embedding vector;
subtracting the first target patient embedding vector and the second target patient embedding vector from a baseline;
selecting the first target patient embedding vector or the second target patient embedding vector based on the first time period or the second time period, respectively, being a predefined time from a health event experience by a reference patient; and
converting the first target patient embedding vector and the second target patient embedding vector to a temporal embedding vector using a machine learning (ML) model.
